SEQUENCE LISTING (17 sequences)

SEQ ID NO: 1 - protein, 2641 aa, Artificial
  sorLA-reporter construct
  MISC_FEATURE (1)..(2641): sorLA-reporter construct
MLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGLESAALQPEPIKVYGQVSLNDSHNQMVVHWAGEKSNVIVALARDSLALARPKSSDVYVSYDYGKSFKKISDKLNFGLGNRSEAVIAQFYHSPADNKRYIFADAYAQYLWITFDFCNTLQGFSIPFRAADLLLHSKASNLLLGFDRSHPNKQLWKSDDFGQTWIMIQEHVKSFSWGIDPYDKPNTIYIERHEPSGYSTVFRSTDFFQSRENQEVILEEVRDFQLRDKYMFATKVVHLLGSEQQSSVQLWVSFGRKPMRAAQFVTRHPINEYYIADASEDQVFVCVSHSNNRTNLYISEAEGLKFSLSLENVLYYSPGGAGSDTLVRYFANEPFADFHRVEGLQGVYIATLINGSMNEENMRSVITFDKGGTWEFLQAPAFTGYGEKINCELSQGCSLHLAQRLSQLLNLQLRRMPILSKESAPGLIIATGSVGKNLASKTNVYISSSAGARWREALPGPHYYTWGDHGGIITAIAQGMETNELKYSTNEGETWKTFIFSEKPVFVYGLLTEPGEKSTVFTIFGSNKENVHSWLILQVNATDALGVPCTENDYKLWSPSDERGNECLLGHKTVFKRRTPHATCFNGEDFDRPVVVSNCSCTREDYECDFGFKMSEDLSLEVCVPDPEFSGKSYSPPVPCPVGSTYRRTRGYRKISGDTCSGGDVEARLEGELVPCPLAEENEFILYAVRKSIYRYDLASGATEQLPLTGLRAAVALDFDYEHNCLYWSDLALDVIQRLCLNGSTGQEVIINSGLETVEALAFEPLSQLLYWVDAGFKKIEVANPDGDFRLTIVNSSVLDRPRALVLVPQEGVMFWTDWGDLKPGIYRSNMDGSAAYHLVSEDVKWPNGISVDDQWIYWTDAYLECIERITFSGQQRSVILDNLPHPYAIAVFKNEIYWDDWSQLSIFRASKYSGSQMEILANQLTGLMDMKIFYKGKNTGSNACVPRPCSLLCLPKANNSRSCRCPEDVSSSVLPSGDLMCDCPQGYQLKNNTCVKQENTCLRNQYRCSNGNCINSIWWCDFDNDCGDMSDERNCPTTICDLDTQFRCQESGTCIPLSYKCDLEDDCGDNSDESHCEMHQCRSDEYNCSSGMCIRSSWVCDGDNDCRDWSDEANCTAIYHTCEASNFQCRNGHCIPQRWACDGDTDCQDGSDEDPVNCEKKCNGFRCPNGTCIPSSKHCDGLRDCSDGSDEQHCEPLCTHFMDFVCKNRQQCLFHSMVCDGIIQCRDGSDEDAAFAGCSQDPEFHKVCDEFGFQCQNGVCISLIWKCDGMDDCGDYSDEANCENPTEAPNCSRYFQFRCENGHCIPNRWKCDRENDCGDWSDEKDCGDSHILPFSTPGPSTCLPNYYRCSSGTCVMDTWVCDGYRDCADGSDEEACPLLANVTAASTPTQLGRCDRFEFECHQPKTCIPNWKRCDGHQDCQDGRDEANCPTHSTLTCMSREFQCEDGEACIVLSERCDGFLDCSDESDEKACSDELTVYKVQNLQWTADFSGDVTLTWMRPKKMPSASCVYNVYYRVVGESIWKTLETHSNKTNTVLKVLKPDTTYQVKVQVQCLSKAHNTNDFVTLRTPEGLPDAPRNLQLSLPREAEGVIVGHWAPPIHTHGLIREYIVEYSRSGSKMWASQRAASNFTEIKNLLVNTLYTVRVAAVTSRGIGNWSDSKSITTIKGKVIPPPDIHIDSYGENYLSFTLTMESDIKVNGYVVNLFWAFDTHKQERRTLNFRGSILSHKVGNLTAHTSYEISAWAKTDLGDSPLAFEHVMTRGVRPPAPSLKAKAINQTAVECTWTGPRNVVYGIFYATSFLDLYRNPKSLTTSLHNKTVIVSKDEQYLFLVRVVVPYQGPSSDYVVVKMIPDSRLPPRHLHVVHTGKTSVVIKWESPYDSPDQDLLYAVAVKDLIRKTDRSYKVKSRNSTVEYTLNKLEPGGKYHIIVQLGNMSKDSSIKITTVSLSAPDALKIITENDHVLLFWKSLALKEKHFNESRGYEIHMFDSAMNITAYLGNTTDNFFKISNLKMGHNYTFTVQARCLFGNQICGEPAILLYDELGSGADASATQAARSTDVAAVVVPILFLILLSLGVGFAILYTKHRRLQSSFTAFANSHYSSRLGSAIFSSGDDLGEDDEDAPMITGFSDDVPMVIA

SEQ ID NO: 2 - protein, 2644 aa, Artificial
  sorLA-reporter construct
  MISC_FEATURE (1)..(2644): sorLA-reporter construct
MLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGLSRSESAALQPEPIKVYGQVSLNDSHNQMVVHWAGEKSNVIVALARDSLALARPKSSDVYVSYDYGKSFKKISDKLNFGLGNRSEAVIAQFYHSPADNKRYIFADAYAQYLWITFDFCNTLQGFSIPFRAADLLLHSKASNLLLGFDRSHPNKQLWKSDDFGQTWIMIQEHVKSFSWGIDPYDKPNTIYIERHEPSGYSTVFRSTDFFQSRENQEVILEEVRDFQLRDKYMFATKVVHLLGSEQQSSVQLWVSFGRKPMRAAQFVTRHPINEYYIADASEDQVFVCVSHSNNRTNLYISEAEGLKFSLSLENVLYYSPGGAGSDTLVRYFANEPFADFHRVEGLQGVYIATLINGSMNEENMRSVITFDKGGTWEFLQAPAFTGYGEKINCELSQGCSLHLAQRLSQLLNLQLRRMPILSKESAPGLIIATGSVGKNLASKTNVYISSSAGARWREALPGPHYYTWGDHGGIITAIAQGMETNELKYSTNEGETWKTFIFSEKPVFVYGLLTEPGEKSTVFTIFGSNKENVHSWLILQVNATDALGVPCTENDYKLWSPSDERGNECLLGHKTVFKRRTPHATCFNGEDFDRPVVVSNCSCTREDYECDFGFKMSEDLSLEVCVPDPEFSGKSYSPPVPCPVGSTYRRTRGYRKISGDTCSGGDVEARLEGELVPCPLAEENEFILYAVRKSIYRYDLASGATEQLPLTGLRAAVALDFDYEHNCLYWSDLALDVIQRLCLNGSTGQEVIINSGLETVEALAFEPLSQLLYWVDAGFKKIEVANPDGDFRLTIVNSSVLDRPRALVLVPQEGVMFWTDWGDLKPGIYRSNMDGSAAYHLVSEDVKWPNGISVDDQWIYWTDAYLECIERITFSGQQRSVILDNLPHPYAIAVFKNEIYWDDWSQLSIFRASKYSGSQMEILANQLTGLMDMKIFYKGKNTGSNACVPRPCSLLCLPKANNSRSCRCPEDVSSSVLPSGDLMCDCPQGYQLKNNTCVKQENTCLRNQYRCSNGNCINSIWWCDFDNDCGDMSDERNCPTTICDLDTQFRCQESGTCIPLSYKCDLEDDCGDNSDESHCEMHQCRSDEYNCSSGMCIRSSWVCDGDNDCRDWSDEANCTAIYHTCEASNFQCRNGHCIPQRWACDGDTDCQDGSDEDPVNCEKKCNGFRCPNGTCIPSSKHCDGLRDCSDGSDEQHCEPLCTHFMDFVCKNRQQCLFHSMVCDGIIQCRDGSDEDAAFAGCSQDPEFHKVCDEFGFQCQNGVCISLIWKCDGMDDCGDYSDEANCENPTEAPNCSRYFQFRCENGHCIPNRWKCDRENDCGDWSDEKDCGDSHILPFSTPGPSTCLPNYYRCSSGTCVMDTWVCDGYRDCADGSDEEACPLLANVTAASTPTQLGRCDRFEFECHQPKTCIPNWKRCDGHQDCQDGRDEANCPTHSTLTCMSREFQCEDGEACIVLSERCDGFLDCSDESDEKACSDELTVYKVQNLQWTADFSGDVTLTWMRPKKMPSASCVYNVYYRVVGESIWKTLETHSNKTNTVLKVLKPDTTYQVKVQVQCLSKAHNTNDFVTLRTPEGLPDAPRNLQLSLPREAEGVIVGHWAPPIHTHGLIREYIVEYSRSGSKMWASQRAASNFTEIKNLLVNTLYTVRVAAVTSRGIGNWSDSKSITTIKGKVIPPPDIHIDSYGENYLSFTLTMESDIKVNGYVVNLFWAFDTHKQERRTLNFRGSILSHKVGNLTAHTSYEISAWAKTDLGDSPLAFEHVMTRGVRPPAPSLKAKAINQTAVECTWTGPRNVVYGIFYATSFLDLYRNPKSLTTSLHNKTVIVSKDEQYLFLVRVVVPYQGPSSDYVVVKMIPDSRLPPRHLHVVHTGKTSVVIKWESPYDSPDQDLLYAVAVKDLIRKTDRSYKVKSRNSTVEYTLNKLEPGGKYHIIVQLGNMSKDSSIKITTVSLSAPDALKIITENDHVLLFWKSLALKEKHFNESRGYEIHMFDSAMNITAYLGNTTDNFFKISNLKMGHNYTFTVQARCLFGNQICGEPAILLYDELGSGADASATQAARSTDVAAVVVPILFLILLSLGVGFAILYTKHRRLQSSFTAFANSHYSSRLGSAIFSSGDDLGEDDEDAPMITGFSDDVPMVIA

SEQ ID NO: 3 - protein, 2647 aa, Artificial
  sorLA-reporter construct
  MISC_FEATURE (1)..(2647): sorLA-reporter construct
MLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGLSRSSGGESAALQPEPIKVYGQVSLNDSHNQMVVHWAGEKSNVIVALARDSLALARPKSSDVYVSYDYGKSFKKISDKLNFGLGNRSEAVIAQFYHSPADNKRYIFADAYAQYLWITFDFCNTLQGFSIPFRAADLLLHSKASNLLLGFDRSHPNKQLWKSDDFGQTWIMIQEHVKSFSWGIDPYDKPNTIYIERHEPSGYSTVFRSTDFFQSRENQEVILEEVRDFQLRDKYMFATKVVHLLGSEQQSSVQLWVSFGRKPMRAAQFVTRHPINEYYIADASEDQVFVCVSHSNNRTNLYISEAEGLKFSLSLENVLYYSPGGAGSDTLVRYFANEPFADFHRVEGLQGVYIATLINGSMNEENMRSVITFDKGGTWEFLQAPAFTGYGEKINCELSQGCSLHLAQRLSQLLNLQLRRMPILSKESAPGLIIATGSVGKNLASKTNVYISSSAGARWREALPGPHYYTWGDHGGIITAIAQGMETNELKYSTNEGETWKTFIFSEKPVFVYGLLTEPGEKSTVFTIFGSNKENVHSWLILQVNATDALGVPCTENDYKLWSPSDERGNECLLGHKTVFKRRTPHATCFNGEDFDRPVVVSNCSCTREDYECDFGFKMSEDLSLEVCVPDPEFSGKSYSPPVPCPVGSTYRRTRGYRKISGDTCSGGDVEARLEGELVPCPLAEENEFILYAVRKSIYRYDLASGATEQLPLTGLRAAVALDFDYEHNCLYWSDLALDVIQRLCLNGSTGQEVIINSGLETVEALAFEPLSQLLYWVDAGFKKIEVANPDGDFRLTIVNSSVLDRPRALVLVPQEGVMFWTDWGDLKPGIYRSNMDGSAAYHLVSEDVKWPNGISVDDQWIYWTDAYLECIERITFSGQQRSVILDNLPHPYAIAVFKNEIYWDDWSQLSIFRASKYSGSQMEILANQLTGLMDMKIFYKGKNTGSNACVPRPCSLLCLPKANNSRSCRCPEDVSSSVLPSGDLMCDCPQGYQLKNNTCVKQENTCLRNQYRCSNGNCINSIWWCDFDNDCGDMSDERNCPTTICDLDTQFRCQESGTCIPLSYKCDLEDDCGDNSDESHCEMHQCRSDEYNCSSGMCIRSSWVCDGDNDCRDWSDEANCTAIYHTCEASNFQCRNGHCIPQRWACDGDTDCQDGSDEDPVNCEKKCNGFRCPNGTCIPSSKHCDGLRDCSDGSDEQHCEPLCTHFMDFVCKNRQQCLFHSMVCDGIIQCRDGSDEDAAFAGCSQDPEFHKVCDEFGFQCQNGVCISLIWKCDGMDDCGDYSDEANCENPTEAPNCSRYFQFRCENGHCIPNRWKCDRENDCGDWSDEKDCGDSHILPFSTPGPSTCLPNYYRCSSGTCVMDTWVCDGYRDCADGSDEEACPLLANVTAASTPTQLGRCDRFEFECHQPKTCIPNWKRCDGHQDCQDGRDEANCPTHSTLTCMSREFQCEDGEACIVLSERCDGFLDCSDESDEKACSDELTVYKVQNLQWTADFSGDVTLTWMRPKKMPSASCVYNVYYRVVGESIWKTLETHSNKTNTVLKVLKPDTTYQVKVQVQCLSKAHNTNDFVTLRTPEGLPDAPRNLQLSLPREAEGVIVGHWAPPIHTHGLIREYIVEYSRSGSKMWASQRAASNFTEIKNLLVNTLYTVRVAAVTSRGIGNWSDSKSITTIKGKVIPPPDIHIDSYGENYLSFTLTMESDIKVNGYVVNLFWAFDTHKQERRTLNFRGSILSHKVGNLTAHTSYEISAWAKTDLGDSPLAFEHVMTRGVRPPAPSLKAKAINQTAVECTWTGPRNVVYGIFYATSFLDLYRNPKSLTTSLHNKTVIVSKDEQYLFLVRVVVPYQGPSSDYVVVKMIPDSRLPPRHLHVVHTGKTSVVIKWESPYDSPDQDLLYAVAVKDLIRKTDRSYKVKSRNSTVEYTLNKLEPGGKYHIIVQLGNMSKDSSIKITTVSLSAPDALKIITENDHVLLFWKSLALKEKHFNESRGYEIHMFDSAMNITAYLGNTTDNFFKISNLKMGHNYTFTVQARCLFGNQICGEPAILLYDELGSGADASATQAARSTDVAAVVVPILFLILLSLGVGFAILYTKHRRLQSSFTAFANSHYSSRLGSAIFSSGDDLGEDDEDAPMITGFSDDVPMVIA

SEQ ID NO: 4 - protein, 2692 aa, Artificial
  sorLA-reporter construct
  MISC_FEATURE (1)..(2692): sorLA-reporter construct
MLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGEVWTQRLHGGSAPLPQDRGFLVVQGDPRELRLWARGDARGASRADEKPLGSGSSAALQPEPIKVYGQVSLNDSHNQMVVHWAGEKSNVIVALARDSLALARPKSSDVYVSYDYGKSFKKISDKLNFGLGNRSEAVIAQFYHSPADNKRYIFADAYAQYLWITFDFCNTLQGFSIPFRAADLLLHSKASNLLLGFDRSHPNKQLWKSDDFGQTWIMIQEHVKSFSWGIDPYDKPNTIYIERHEPSGYSTVFRSTDFFQSRENQEVILEEVRDFQLRDKYMFATKVVHLLGSEQQSSVQLWVSFGRKPMRAAQFVTRHPINEYYIADASEDQVFVCVSHSNNRTNLYISEAEGLKFSLSLENVLYYSPGGAGSDTLVRYFANEPFADFHRVEGLQGVYIATLINGSMNEENMRSVITFDKGGTWEFLQAPAFTGYGEKINCELSQGCSLHLAQRLSQLLNLQLRRMPILSKESAPGLIIATGSVGKNLASKTNVYISSSAGARWREALPGPHYYTWGDHGGIITAIAQGMETNELKYSTNEGETWKTFIFSEKPVFVYGLLTEPGEKSTVFTIFGSNKENVHSWLILQVNATDALGVPCTENDYKLWSPSDERGNECLLGHKTVFKRRTPHATCFNGEDFDRPVVVSNCSCTREDYECDFGFKMSEDLSLEVCVPDPEFSGKSYSPPVPCPVGSTYRRTRGYRKISGDTCSGGDVEARLEGELVPCPLAEENEFILYAVRKSIYRYDLASGATEQLPLTGLRAAVALDFDYEHNCLYWSDLALDVIQRLCLNGSTGQEVIINSGLETVEALAFEPLSQLLYWVDAGFKKIEVANPDGDFRLTIVNSSVLDRPRALVLVPQEGVMFWTDWGDLKPGIYRSNMDGSAAYHLVSEDVKWPNGISVDDQWIYWTDAYLECIERITFSGQQRSVILDNLPHPYAIAVFKNEIYWDDWSQLSIFRASKYSGSQMEILANQLTGLMDMKIFYKGKNTGSNACVPRPCSLLCLPKANNSRSCRCPEDVSSSVLPSGDLMCDCPQGYQLKNNTCVKQENTCLRNQYRCSNGNCINSIWWCDFDNDCGDMSDERNCPTTICDLDTQFRCQESGTCIPLSYKCDLEDDCGDNSDESHCEMHQCRSDEYNCSSGMCIRSSWVCDGDNDCRDWSDEANCTAIYHTCEASNFQCRNGHCIPQRWACDGDTDCQDGSDEDPVNCEKKCNGFRCPNGTCIPSSKHCDGLRDCSDGSDEQHCEPLCTHFMDFVCKNRQQCLFHSMVCDGIIQCRDGSDEDAAFAGCSQDPEFHKVCDEFGFQCQNGVCISLIWKCDGMDDCGDYSDEANCENPTEAPNCSRYFQFRCENGHCIPNRWKCDRENDCGDWSDEKDCGDSHILPFSTPGPSTCLPNYYRCSSGTCVMDTWVCDGYRDCADGSDEEACPLLANVTAASTPTQLGRCDRFEFECHQPKTCIPNWKRCDGHQDCQDGRDEANCPTHSTLTCMSREFQCEDGEACIVLSERCDGFLDCSDESDEKACSDELTVYKVQNLQWTADFSGDVTLTWMRPKKMPSASCVYNVYYRVVGESIWKTLETHSNKTNTVLKVLKPDTTYQVKVQVQCLSKAHNTNDFVTLRTPEGLPDAPRNLQLSLPREAEGVIVGHWAPPIHTHGLIREYIVEYSRSGSKMWASQRAASNFTEIKNLLVNTLYTVRVAAVTSRGIGNWSDSKSITTIKGKVIPPPDIHIDSYGENYLSFTLTMESDIKVNGYVVNLFWAFDTHKQERRTLNFRGSILSHKVGNLTAHTSYEISAWAKTDLGDSPLAFEHVMTRGVRPPAPSLKAKAINQTAVECTWTGPRNVVYGIFYATSFLDLYRNPKSLTTSLHNKTVIVSKDEQYLFLVRVVVPYQGPSSDYVVVKMIPDSRLPPRHLHVVHTGKTSVVIKWESPYDSPDQDLLYAVAVKDLIRKTDRSYKVKSRNSTVEYTLNKLEPGGKYHIIVQLGNMSKDSSIKITTVSLSAPDALKIITENDHVLLFWKSLALKEKHFNESRGYEIHMFDSAMNITAYLGNTTDNFFKISNLKMGHNYTFTVQARCLFGNQICGEPAILLYDELGSGADASATQAARSTDVAAVVVPILFLILLSLGVGFAILYTKHRRLQSSFTAFANSHYSSRLGSAIFSSGDDLGEDDEDAPMITGFSDDVPMVIA

SEQ ID NO: 5 - protein, 2639 aa, Artificial
  sorLA-reporter construct
  MISC_FEATURE (1)..(2639): sorLA-reporter construct
MLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGSAALQPEPIKVYGQVSLNDSHNQMVVHWAGEKSNVIVALARDSLALARPKSSDVYVSYDYGKSFKKISDKLNFGLGNRSEAVIAQFYHSPADNKRYIFADAYAQYLWITFDFCNTLQGFSIPFRAADLLLHSKASNLLLGFDRSHPNKQLWKSDDFGQTWIMIQEHVKSFSWGIDPYDKPNTIYIERHEPSGYSTVFRSTDFFQSRENQEVILEEVRDFQLRDKYMFATKVVHLLGSEQQSSVQLWVSFGRKPMRAAQFVTRHPINEYYIADASEDQVFVCVSHSNNRTNLYISEAEGLKFSLSLENVLYYSPGGAGSDTLVRYFANEPFADFHRVEGLQGVYIATLINGSMNEENMRSVITFDKGGTWEFLQAPAFTGYGEKINCELSQGCSLHLAQRLSQLLNLQLRRMPILSKESAPGLIIATGSVGKNLASKTNVYISSSAGARWREALPGPHYYTWGDHGGIITAIAQGMETNELKYSTNEGETWKTFIFSEKPVFVYGLLTEPGEKSTVFTIFGSNKENVHSWLILQVNATDALGVPCTENDYKLWSPSDERGNECLLGHKTVFKRRTPHATCFNGEDFDRPVVVSNCSCTREDYECDFGFKMSEDLSLEVCVPDPEFSGKSYSPPVPCPVGSTYRRTRGYRKISGDTCSGGDVEARLEGELVPCPLAEENEFILYAVRKSIYRYDLASGATEQLPLTGLRAAVALDFDYEHNCLYWSDLALDVIQRLCLNGSTGQEVIINSGLETVEALAFEPLSQLLYWVDAGFKKIEVANPDGDFRLTIVNSSVLDRPRALVLVPQEGVMFWTDWGDLKPGIYRSNMDGSAAYHLVSEDVKWPNGISVDDQWIYWTDAYLECIERITFSGQQRSVILDNLPHPYAIAVFKNEIYWDDWSQLSIFRASKYSGSQMEILANQLTGLMDMKIFYKGKNTGSNACVPRPCSLLCLPKANNSRSCRCPEDVSSSVLPSGDLMCDCPQGYQLKNNTCVKQENTCLRNQYRCSNGNCINSIWWCDFDNDCGDMSDERNCPTTICDLDTQFRCQESGTCIPLSYKCDLEDDCGDNSDESHCEMHQCRSDEYNCSSGMCIRSSWVCDGDNDCRDWSDEANCTAIYHTCEASNFQCRNGHCIPQRWACDGDTDCQDGSDEDPVNCEKKCNGFRCPNGTCIPSSKHCDGLRDCSDGSDEQHCEPLCTHFMDFVCKNRQQCLFHSMVCDGIIQCRDGSDEDAAFAGCSQDPEFHKVCDEFGFQCQNGVCISLIWKCDGMDDCGDYSDEANCENPTEAPNCSRYFQFRCENGHCIPNRWKCDRENDCGDWSDEKDCGDSHILPFSTPGPSTCLPNYYRCSSGTCVMDTWVCDGYRDCADGSDEEACPLLANVTAASTPTQLGRCDRFEFECHQPKTCIPNWKRCDGHQDCQDGRDEANCPTHSTLTCMSREFQCEDGEACIVLSERCDGFLDCSDESDEKACSDELTVYKVQNLQWTADFSGDVTLTWMRPKKMPSASCVYNVYYRVVGESIWKTLETHSNKTNTVLKVLKPDTTYQVKVQVQCLSKAHNTNDFVTLRTPEGLPDAPRNLQLSLPREAEGVIVGHWAPPIHTHGLIREYIVEYSRSGSKMWASQRAASNFTEIKNLLVNTLYTVRVAAVTSRGIGNWSDSKSITTIKGKVIPPPDIHIDSYGENYLSFTLTMESDIKVNGYVVNLFWAFDTHKQERRTLNFRGSILSHKVGNLTAHTSYEISAWAKTDLGDSPLAFEHVMTRGVRPPAPSLKAKAINQTAVECTWTGPRNVVYGIFYATSFLDLYRNPKSLTTSLHNKTVIVSKDEQYLFLVRVVVPYQGPSSDYVVVKMIPDSRLPPRHLHVVHTGKTSVVIKWESPYDSPDQDLLYAVAVKDLIRKTDRSYKVKSRNSTVEYTLNKLEPGGKYHIIVQLGNMSKDSSIKITTVSLSAPDALKIITENDHVLLFWKSLALKEKHFNESRGYEIHMFDSAMNITAYLGNTTDNFFKISNLKMGHNYTFTVQARCLFGNQICGEPAILLYDELGSGADASATQAARSTDVAAVVVPILFLILLSLGVGFAILYTKHRRLQSSFTAFANSHYSSRLGSAIFSSGDDLGEDDEDAPMITGFSDDVPMVIA

SEQ ID NO: 6 - protein, 2303 aa, Artificial
  sorLA-reporter construct
  MISC_FEATURE (1)..(2303): sorLA-reporter construct
KPTENNEDFNIVAVASNFATTDLDADRGKLPGKKLPLEVLKEIEANARKAGCTRGCLICLSHIKCTPKMKKFIPGRCHTYEGDKESAQGGIGEAIVDIPEIPGFKDLEPIEQFIAQVDLCVDCTTGCLKGLANVQCSDLLKKWLPQRCATFASKIQGQVDKIKGAGGDLESAALQPEPIKVYGQVSLNDSHNQMVVHWAGEKSNVIVALARDSLALARPKSSDVYVSYDYGKSFKKISDKLNFGLGNRSEAVIAQFYHSPADNKRYIFADAYAQYLWITFDFCNTLQGFSIPFRAADLLLHSKASNLLLGFDRSHPNKQLWKSDDFGQTWIMIQEHVKSFSWGIDPYDKPNTIYIERHEPSGYSTVFRSTDFFQSRENQEVILEEVRDFQLRDKYMFATKVVHLLGSEQQSSVQLWVSFGRKPMRAAQFVTRHPINEYYIADASEDQVFVCVSHSNNRTNLYISEAEGLKFSLSLENVLYYSPGGAGSDTLVRYFANEPFADFHRVEGLQGVYIATLINGSMNEENMRSVITFDKGGTWEFLQAPAFTGYGEKINCELSQGCSLHLAQRLSQLLNLQLRRMPILSKESAPGLIIATGSVGKNLASKTNVYISSSAGARWREALPGPHYYTWGDHGGIITAIAQGMETNELKYSTNEGETWKTFIFSEKPVFVYGLLTEPGEKSTVFTIFGSNKENVHSWLILQVNATDALGVPCTENDYKLWSPSDERGNECLLGHKTVFKRRTPHATCFNGEDFDRPVVVSNCSCTREDYECDFGFKMSEDLSLEVCVPDPEFSGKSYSPPVPCPVGSTYRRTRGYRKISGDTCSGGDVEARLEGELVPCPLAEENEFILYAVRKSIYRYDLASGATEQLPLTGLRAAVALDFDYEHNCLYWSDLALDVIQRLCLNGSTGQEVIINSGLETVEALAFEPLSQLLYWVDAGFKKIEVANPDGDFRLTIVNSSVLDRPRALVLVPQEGVMFWTDWGDLKPGIYRSNMDGSAAYHLVSEDVKWPNGISVDDQWIYWTDAYLECIERITFSGQQRSVILDNLPHPYAIAVFKNEIYWDDWSQLSIFRASKYSGSQMEILANQLTGLMDMKIFYKGKNTGSNACVPRPCSLLCLPKANNSRSCRCPEDVSSSVLPSGDLMCDCPQGYQLKNNTCVKQENTCLRNQYRCSNGNCINSIWWCDFDNDCGDMSDERNCPTTICDLDTQFRCQESGTCIPLSYKCDLEDDCGDNSDESHCEMHQCRSDEYNCSSGMCIRSSWVCDGDNDCRDWSDEANCTAIYHTCEASNFQCRNGHCIPQRWACDGDTDCQDGSDEDPVNCEKKCNGFRCPNGTCIPSSKHCDGLRDCSDGSDEQHCEPLCTHFMDFVCKNRQQCLFHSMVCDGIIQCRDGSDEDAAFAGCSQDPEFHKVCDEFGFQCQNGVCISLIWKCDGMDDCGDYSDEANCENPTEAPNCSRYFQFRCENGHCIPNRWKCDRENDCGDWSDEKDCGDSHILPFSTPGPSTCLPNYYRCSSGTCVMDTWVCDGYRDCADGSDEEACPLLANVTAASTPTQLGRCDRFEFECHQPKTCIPNWKRCDGHQDCQDGRDEANCPTHSTLTCMSREFQCEDGEACIVLSERCDGFLDCSDESDEKACSDELTVYKVQNLQWTADFSGDVTLTWMRPKKMPSASCVYNVYYRVVGESIWKTLETHSNKTNTVLKVLKPDTTYQVKVQVQCLSKAHNTNDFVTLRTPEGLPDAPRNLQLSLPREAEGVIVGHWAPPIHTHGLIREYIVEYSRSGSKMWASQRAASNFTEIKNLLVNTLYTVRVAAVTSRGIGNWSDSKSITTIKGKVIPPPDIHIDSYGENYLSFTLTMESDIKVNGYVVNLFWAFDTHKQERRTLNFRGSILSHKVGNLTAHTSYEISAWAKTDLGDSPLAFEHVMTRGVRPPAPSLKAKAINQTAVECTWTGPRNVVYGIFYATSFLDLYRNPKSLTTSLHNKTVIVSKDEQYLFLVRVVVPYQGPSSDYVVVKMIPDSRLPPRHLHVVHTGKTSVVIKWESPYDSPDQDLLYAVAVKDLIRKTDRSYKVKSRNSTVEYTLNKLEPGGKYHIIVQLGNMSKDSSIKITTVSLSAPDALKIITENDHVLLFWKSLALKEKHFNESRGYEIHMFDSAMNITAYLGNTTDNFFKISNLKMGHNYTFTVQARCLFGNQICGEPAILLYDELGSGADASATQAARSTDVAAVVVPILFLILLSLGVGFAILYTKHRRLQSSFTAFANSHYSSRLGSAIFSSGDDLGEDDEDAPMITGFSDDVPMVIA

SEQ ID NO: 7 - protein, 2306 aa, Artificial
  sorLA-reporter construct
  MISC_FEATURE (1)..(2306): sorLA-reporter construct
KPTENNEDFNIVAVASNFATTDLDADRGKLPGKKLPLEVLKEIEANARKAGCTRGCLICLSHIKCTPKMKKFIPGRCHTYEGDKESAQGGIGEAIVDIPEIPGFKDLEPIEQFIAQVDLCVDCTTGCLKGLANVQCSDLLKKWLPQRCATFASKIQGQVDKIKGAGGDLSRSESAALQPEPIKVYGQVSLNDSHNQMVVHWAGEKSNVIVALARDSLALARPKSSDVYVSYDYGKSFKKISDKLNFGLGNRSEAVIAQFYHSPADNKRYIFADAYAQYLWITFDFCNTLQGFSIPFRAADLLLHSKASNLLLGFDRSHPNKQLWKSDDFGQTWIMIQEHVKSFSWGIDPYDKPNTIYIERHEPSGYSTVFRSTDFFQSRENQEVILEEVRDFQLRDKYMFATKVVHLLGSEQQSSVQLWVSFGRKPMRAAQFVTRHPINEYYIADASEDQVFVCVSHSNNRTNLYISEAEGLKFSLSLENVLYYSPGGAGSDTLVRYFANEPFADFHRVEGLQGVYIATLINGSMNEENMRSVITFDKGGTWEFLQAPAFTGYGEKINCELSQGCSLHLAQRLSQLLNLQLRRMPILSKESAPGLIIATGSVGKNLASKTNVYISSSAGARWREALPGPHYYTWGDHGGIITAIAQGMETNELKYSTNEGETWKTFIFSEKPVFVYGLLTEPGEKSTVFTIFGSNKENVHSWLILQVNATDALGVPCTENDYKLWSPSDERGNECLLGHKTVFKRRTPHATCFNGEDFDRPVVVSNCSCTREDYECDFGFKMSEDLSLEVCVPDPEFSGKSYSPPVPCPVGSTYRRTRGYRKISGDTCSGGDVEARLEGELVPCPLAEENEFILYAVRKSIYRYDLASGATEQLPLTGLRAAVALDFDYEHNCLYWSDLALDVIQRLCLNGSTGQEVIINSGLETVEALAFEPLSQLLYWVDAGFKKIEVANPDGDFRLTIVNSSVLDRPRALVLVPQEGVMFWTDWGDLKPGIYRSNMDGSAAYHLVSEDVKWPNGISVDDQWIYWTDAYLECIERITFSGQQRSVILDNLPHPYAIAVFKNEIYWDDWSQLSIFRASKYSGSQMEILANQLTGLMDMKIFYKGKNTGSNACVPRPCSLLCLPKANNSRSCRCPEDVSSSVLPSGDLMCDCPQGYQLKNNTCVKQENTCLRNQYRCSNGNCINSIWWCDFDNDCGDMSDERNCPTTICDLDTQFRCQESGTCIPLSYKCDLEDDCGDNSDESHCEMHQCRSDEYNCSSGMCIRSSWVCDGDNDCRDWSDEANCTAIYHTCEASNFQCRNGHCIPQRWACDGDTDCQDGSDEDPVNCEKKCNGFRCPNGTCIPSSKHCDGLRDCSDGSDEQHCEPLCTHFMDFVCKNRQQCLFHSMVCDGIIQCRDGSDEDAAFAGCSQDPEFHKVCDEFGFQCQNGVCISLIWKCDGMDDCGDYSDEANCENPTEAPNCSRYFQFRCENGHCIPNRWKCDRENDCGDWSDEKDCGDSHILPFSTPGPSTCLPNYYRCSSGTCVMDTWVCDGYRDCADGSDEEACPLLANVTAASTPTQLGRCDRFEFECHQPKTCIPNWKRCDGHQDCQDGRDEANCPTHSTLTCMSREFQCEDGEACIVLSERCDGFLDCSDESDEKACSDELTVYKVQNLQWTADFSGDVTLTWMRPKKMPSASCVYNVYYRVVGESIWKTLETHSNKTNTVLKVLKPDTTYQVKVQVQCLSKAHNTNDFVTLRTPEGLPDAPRNLQLSLPREAEGVIVGHWAPPIHTHGLIREYIVEYSRSGSKMWASQRAASNFTEIKNLLVNTLYTVRVAAVTSRGIGNWSDSKSITTIKGKVIPPPDIHIDSYGENYLSFTLTMESDIKVNGYVVNLFWAFDTHKQERRTLNFRGSILSHKVGNLTAHTSYEISAWAKTDLGDSPLAFEHVMTRGVRPPAPSLKAKAINQTAVECTWTGPRNVVYGIFYATSFLDLYRNPKSLTTSLHNKTVIVSKDEQYLFLVRVVVPYQGPSSDYVVVKMIPDSRLPPRHLHVVHTGKTSVVIKWESPYDSPDQDLLYAVAVKDLIRKTDRSYKVKSRNSTVEYTLNKLEPGGKYHIIVQLGNMSKDSSIKITTVSLSAPDALKIITENDHVLLFWKSLALKEKHFNESRGYEIHMFDSAMNITAYLGNTTDNFFKISNLKMGHNYTFTVQARCLFGNQICGEPAILLYDELGSGADASATQAARSTDVAAVVVPILFLILLSLGVGFAILYTKHRRLQSSFTAFANSHYSSRLGSAIFSSGDDLGEDDEDAPMITGFSDDVPMVIA

SEQ ID NO: 8 - protein, 2309 aa, Artificial
  sorLA-reporter construct
  MISC_FEATURE (1)..(2309): sorLA-reporter construct
KPTENNEDFNIVAVASNFATTDLDADRGKLPGKKLPLEVLKEIEANARKAGCTRGCLICLSHIKCTPKMKKFIPGRCHTYEGDKESAQGGIGEAIVDIPEIPGFKDLEPIEQFIAQVDLCVDCTTGCLKGLANVQCSDLLKKWLPQRCATFASKIQGQVDKIKGAGGDLSRSSGGESAALQPEPIKVYGQVSLNDSHNQMVVHWAGEKSNVIVALARDSLALARPKSSDVYVSYDYGKSFKKISDKLNFGLGNRSEAVIAQFYHSPADNKRYIFADAYAQYLWITFDFCNTLQGFSIPFRAADLLLHSKASNLLLGFDRSHPNKQLWKSDDFGQTWIMIQEHVKSFSWGIDPYDKPNTIYIERHEPSGYSTVFRSTDFFQSRENQEVILEEVRDFQLRDKYMFATKVVHLLGSEQQSSVQLWVSFGRKPMRAAQFVTRHPINEYYIADASEDQVFVCVSHSNNRTNLYISEAEGLKFSLSLENVLYYSPGGAGSDTLVRYFANEPFADFHRVEGLQGVYIATLINGSMNEENMRSVITFDKGGTWEFLQAPAFTGYGEKINCELSQGCSLHLAQRLSQLLNLQLRRMPILSKESAPGLIIATGSVGKNLASKTNVYISSSAGARWREALPGPHYYTWGDHGGIITAIAQGMETNELKYSTNEGETWKTFIFSEKPVFVYGLLTEPGEKSTVFTIFGSNKENVHSWLILQVNATDALGVPCTENDYKLWSPSDERGNECLLGHKTVFKRRTPHATCFNGEDFDRPVVVSNCSCTREDYECDFGFKMSEDLSLEVCVPDPEFSGKSYSPPVPCPVGSTYRRTRGYRKISGDTCSGGDVEARLEGELVPCPLAEENEFILYAVRKSIYRYDLASGATEQLPLTGLRAAVALDFDYEHNCLYWSDLALDVIQRLCLNGSTGQEVIINSGLETVEALAFEPLSQLLYWVDAGFKKIEVANPDGDFRLTIVNSSVLDRPRALVLVPQEGVMFWTDWGDLKPGIYRSNMDGSAAYHLVSEDVKWPNGISVDDQWIYWTDAYLECIERITFSGQQRSVILDNLPHPYAIAVFKNEIYWDDWSQLSIFRASKYSGSQMEILANQLTGLMDMKIFYKGKNTGSNACVPRPCSLLCLPKANNSRSCRCPEDVSSSVLPSGDLMCDCPQGYQLKNNTCVKQENTCLRNQYRCSNGNCINSIWWCDFDNDCGDMSDERNCPTTICDLDTQFRCQESGTCIPLSYKCDLEDDCGDNSDESHCEMHQCRSDEYNCSSGMCIRSSWVCDGDNDCRDWSDEANCTAIYHTCEASNFQCRNGHCIPQRWACDGDTDCQDGSDEDPVNCEKKCNGFRCPNGTCIPSSKHCDGLRDCSDGSDEQHCEPLCTHFMDFVCKNRQQCLFHSMVCDGIIQCRDGSDEDAAFAGCSQDPEFHKVCDEFGFQCQNGVCISLIWKCDGMDDCGDYSDEANCENPTEAPNCSRYFQFRCENGHCIPNRWKCDRENDCGDWSDEKDCGDSHILPFSTPGPSTCLPNYYRCSSGTCVMDTWVCDGYRDCADGSDEEACPLLANVTAASTPTQLGRCDRFEFECHQPKTCIPNWKRCDGHQDCQDGRDEANCPTHSTLTCMSREFQCEDGEACIVLSERCDGFLDCSDESDEKACSDELTVYKVQNLQWTADFSGDVTLTWMRPKKMPSASCVYNVYYRVVGESIWKTLETHSNKTNTVLKVLKPDTTYQVKVQVQCLSKAHNTNDFVTLRTPEGLPDAPRNLQLSLPREAEGVIVGHWAPPIHTHGLIREYIVEYSRSGSKMWASQRAASNFTEIKNLLVNTLYTVRVAAVTSRGIGNWSDSKSITTIKGKVIPPPDIHIDSYGENYLSFTLTMESDIKVNGYVVNLFWAFDTHKQERRTLNFRGSILSHKVGNLTAHTSYEISAWAKTDLGDSPLAFEHVMTRGVRPPAPSLKAKAINQTAVECTWTGPRNVVYGIFYATSFLDLYRNPKSLTTSLHNKTVIVSKDEQYLFLVRVVVPYQGPSSDYVVVKMIPDSRLPPRHLHVVHTGKTSVVIKWESPYDSPDQDLLYAVAVKDLIRKTDRSYKVKSRNSTVEYTLNKLEPGGKYHIIVQLGNMSKDSSIKITTVSLSAPDALKIITENDHVLLFWKSLALKEKHFNESRGYEIHMFDSAMNITAYLGNTTDNFFKISNLKMGHNYTFTVQARCLFGNQICGEPAILLYDELGSGADASATQAARSTDVAAVVVPILFLILLSLGVGFAILYTKHRRLQSSFTAFANSHYSSRLGSAIFSSGDDLGEDDEDAPMITGFSDDVPMVIA

SEQ ID NO: 9 - protein, 2354 aa, Artificial
  sorLA-reporter construct
  MISC_FEATURE (1)..(2354): sorLA-reporter construct
KPTENNEDFNIVAVASNFATTDLDADRGKLPGKKLPLEVLKEIEANARKAGCTRGCLICLSHIKCTPKMKKFIPGRCHTYEGDKESAQGGIGEAIVDIPEIPGFKDLEPIEQFIAQVDLCVDCTTGCLKGLANVQCSDLLKKWLPQRCATFASKIQGQVDKIKGAGGDEVWTQRLHGGSAPLPQDRGFLVVQGDPRELRLWARGDARGASRADEKPLGSGSSAALQPEPIKVYGQVSLNDSHNQMVVHWAGEKSNVIVALARDSLALARPKSSDVYVSYDYGKSFKKISDKLNFGLGNRSEAVIAQFYHSPADNKRYIFADAYAQYLWITFDFCNTLQGFSIPFRAADLLLHSKASNLLLGFDRSHPNKQLWKSDDFGQTWIMIQEHVKSFSWGIDPYDKPNTIYIERHEPSGYSTVFRSTDFFQSRENQEVILEEVRDFQLRDKYMFATKVVHLLGSEQQSSVQLWVSFGRKPMRAAQFVTRHPINEYYIADASEDQVFVCVSHSNNRTNLYISEAEGLKFSLSLENVLYYSPGGAGSDTLVRYFANEPFADFHRVEGLQGVYIATLINGSMNEENMRSVITFDKGGTWEFLQAPAFTGYGEKINCELSQGCSLHLAQRLSQLLNLQLRRMPILSKESAPGLIIATGSVGKNLASKTNVYISSSAGARWREALPGPHYYTWGDHGGIITAIAQGMETNELKYSTNEGETWKTFIFSEKPVFVYGLLTEPGEKSTVFTIFGSNKENVHSWLILQVNATDALGVPCTENDYKLWSPSDERGNECLLGHKTVFKRRTPHATCFNGEDFDRPVVVSNCSCTREDYECDFGFKMSEDLSLEVCVPDPEFSGKSYSPPVPCPVGSTYRRTRGYRKISGDTCSGGDVEARLEGELVPCPLAEENEFILYAVRKSIYRYDLASGATEQLPLTGLRAAVALDFDYEHNCLYWSDLALDVIQRLCLNGSTGQEVIINSGLETVEALAFEPLSQLLYWVDAGFKKIEVANPDGDFRLTIVNSSVLDRPRALVLVPQEGVMFWTDWGDLKPGIYRSNMDGSAAYHLVSEDVKWPNGISVDDQWIYWTDAYLECIERITFSGQQRSVILDNLPHPYAIAVFKNEIYWDDWSQLSIFRASKYSGSQMEILANQLTGLMDMKIFYKGKNTGSNACVPRPCSLLCLPKANNSRSCRCPEDVSSSVLPSGDLMCDCPQGYQLKNNTCVKQENTCLRNQYRCSNGNCINSIWWCDFDNDCGDMSDERNCPTTICDLDTQFRCQESGTCIPLSYKCDLEDDCGDNSDESHCEMHQCRSDEYNCSSGMCIRSSWVCDGDNDCRDWSDEANCTAIYHTCEASNFQCRNGHCIPQRWACDGDTDCQDGSDEDPVNCEKKCNGFRCPNGTCIPSSKHCDGLRDCSDGSDEQHCEPLCTHFMDFVCKNRQQCLFHSMVCDGIIQCRDGSDEDAAFAGCSQDPEFHKVCDEFGFQCQNGVCISLIWKCDGMDDCGDYSDEANCENPTEAPNCSRYFQFRCENGHCIPNRWKCDRENDCGDWSDEKDCGDSHILPFSTPGPSTCLPNYYRCSSGTCVMDTWVCDGYRDCADGSDEEACPLLANVTAASTPTQLGRCDRFEFECHQPKTCIPNWKRCDGHQDCQDGRDEANCPTHSTLTCMSREFQCEDGEACIVLSERCDGFLDCSDESDEKACSDELTVYKVQNLQWTADFSGDVTLTWMRPKKMPSASCVYNVYYRVVGESIWKTLETHSNKTNTVLKVLKPDTTYQVKVQVQCLSKAHNTNDFVTLRTPEGLPDAPRNLQLSLPREAEGVIVGHWAPPIHTHGLIREYIVEYSRSGSKMWASQRAASNFTEIKNLLVNTLYTVRVAAVTSRGIGNWSDSKSITTIKGKVIPPPDIHIDSYGENYLSFTLTMESDIKVNGYVVNLFWAFDTHKQERRTLNFRGSILSHKVGNLTAHTSYEISAWAKTDLGDSPLAFEHVMTRGVRPPAPSLKAKAINQTAVECTWTGPRNVVYGIFYATSFLDLYRNPKSLTTSLHNKTVIVSKDEQYLFLVRVVVPYQGPSSDYVVVKMIPDSRLPPRHLHVVHTGKTSVVIKWESPYDSPDQDLLYAVAVKDLIRKTDRSYKVKSRNSTVEYTLNKLEPGGKYHIIVQLGNMSKDSSIKITTVSLSAPDALKIITENDHVLLFWKSLALKEKHFNESRGYEIHMFDSAMNITAYLGNTTDNFFKISNLKMGHNYTFTVQARCLFGNQICGEPAILLYDELGSGADASATQAARSTDVAAVVVPILFLILLSLGVGFAILYTKHRRLQSSFTAFANSHYSSRLGSAIFSSGDDLGEDDEDAPMITGFSDDVPMVIA

SEQ ID NO: 10 - protein, 2133 aa, Homo sapiens
  MISC_FEATURE (1)..(2133): Human sortilin-related receptor 1_sorLA
SAALQPEPIKVYGQVSLNDSHNQMVVHWAGEKSNVIVALARDSLALARPKSSDVYVSYDYGKSFKKISDKLNFGLGNRSEAVIAQFYHSPADNKRYIFADAYAQYLWITFDFCNTLQGFSIPFRAADLLLHSKASNLLLGFDRSHPNKQLWKSDDFGQTWIMIQEHVKSFSWGIDPYDKPNTIYIERHEPSGYSTVFRSTDFFQSRENQEVILEEVRDFQLRDKYMFATKVVHLLGSEQQSSVQLWVSFGRKPMRAAQFVTRHPINEYYIADASEDQVFVCVSHSNNRTNLYISEAEGLKFSLSLENVLYYSPGGAGSDTLVRYFANEPFADFHRVEGLQGVYIATLINGSMNEENMRSVITFDKGGTWEFLQAPAFTGYGEKINCELSQGCSLHLAQRLSQLLNLQLRRMPILSKESAPGLIIATGSVGKNLASKTNVYISSSAGARWREALPGPHYYTWGDHGGIITAIAQGMETNELKYSTNEGETWKTFIFSEKPVFVYGLLTEPGEKSTVFTIFGSNKENVHSWLILQVNATDALGVPCTENDYKLWSPSDERGNECLLGHKTVFKRRTPHATCFNGEDFDRPVVVSNCSCTREDYECDFGFKMSEDLSLEVCVPDPEFSGKSYSPPVPCPVGSTYRRTRGYRKISGDTCSGGDVEARLEGELVPCPLAEENEFILYAVRKSIYRYDLASGATEQLPLTGLRAAVALDFDYEHNCLYWSDLALDVIQRLCLNGSTGQEVIINSGLETVEALAFEPLSQLLYWVDAGFKKIEVANPDGDFRLTIVNSSVLDRPRALVLVPQEGVMFWTDWGDLKPGIYRSNMDGSAAYHLVSEDVKWPNGISVDDQWIYWTDAYLECIERITFSGQQRSVILDNLPHPYAIAVFKNEIYWDDWSQLSIFRASKYSGSQMEILANQLTGLMDMKIFYKGKNTGSNACVPRPCSLLCLPKANNSRSCRCPEDVSSSVLPSGDLMCDCPQGYQLKNNTCVKQENTCLRNQYRCSNGNCINSIWWCDFDNDCGDMSDERNCPTTICDLDTQFRCQESGTCIPLSYKCDLEDDCGDNSDESHCEMHQCRSDEYNCSSGMCIRSSWVCDGDNDCRDWSDEANCTAIYHTCEASNFQCRNGHCIPQRWACDGDTDCQDGSDEDPVNCEKKCNGFRCPNGTCIPSSKHCDGLRDCSDGSDEQHCEPLCTHFMDFVCKNRQQCLFHSMVCDGIIQCRDGSDEDAAFAGCSQDPEFHKVCDEFGFQCQNGVCISLIWKCDGMDDCGDYSDEANCENPTEAPNCSRYFQFRCENGHCIPNRWKCDRENDCGDWSDEKDCGDSHILPFSTPGPSTCLPNYYRCSSGTCVMDTWVCDGYRDCADGSDEEACPLLANVTAASTPTQLGRCDRFEFECHQPKTCIPNWKRCDGHQDCQDGRDEANCPTHSTLTCMSREFQCEDGEACIVLSERCDGFLDCSDESDEKACSDELTVYKVQNLQWTADFSGDVTLTWMRPKKMPSASCVYNVYYRVVGESIWKTLETHSNKTNTVLKVLKPDTTYQVKVQVQCLSKAHNTNDFVTLRTPEGLPDAPRNLQLSLPREAEGVIVGHWAPPIHTHGLIREYIVEYSRSGSKMWASQRAASNFTEIKNLLVNTLYTVRVAAVTSRGIGNWSDSKSITTIKGKVIPPPDIHIDSYGENYLSFTLTMESDIKVNGYVVNLFWAFDTHKQERRTLNFRGSILSHKVGNLTAHTSYEISAWAKTDLGDSPLAFEHVMTRGVRPPAPSLKAKAINQTAVECTWTGPRNVVYGIFYATSFLDLYRNPKSLTTSLHNKTVIVSKDEQYLFLVRVVVPYQGPSSDYVVVKMIPDSRLPPRHLHVVHTGKTSVVIKWESPYDSPDQDLLYAVAVKDLIRKTDRSYKVKSRNSTVEYTLNKLEPGGKYHIIVQLGNMSKDSSIKITTVSLSAPDALKIITENDHVLLFWKSLALKEKHFNESRGYEIHMFDSAMNITAYLGNTTDNFFKISNLKMGHNYTFTVQARCLFGNQICGEPAILLYDELGSGADASATQAARSTDVAAVVVPILFLILLSLGVGFAILYTKHRRLQSSFTAFANSHYSSRLGSAIFSSGDDLGEDDEDAPMITGFSDDVPMVIA

SEQ ID NO: 11 - protein, 506 aa, Artificial
  reporter
  MISC_FEATURE (1)..(506): SEAP reporter
MLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPG

SEQ ID NO: 12 - protein, 5 aa, Artificial
  linker
  MISC_FEATURE (1)..(5): linker
LSRSE

SEQ ID NO: 13 - protein, 8 aa, Artificial
  linker
  MISC_FEATURE (1)..(8): linker
LSRSSGGE

SEQ ID NO: 14 - protein, 53 aa, Artificial
  linker
  MISC_FEATURE (1)..(53): linker
EVWTQRLHGGSAPLPQDRGFLVVQGDPRELRLWARGDARGASRADEKPLGSGS

SEQ ID NO: 15 - protein, 168 aa, Artificial
  reporter
  MISC_FEATURE (1)..(168): eGluc reporter
KPTENNEDFNIVAVASNFATTDLDADRGKLPGKKLPLEVLKEIEANARKAGCTRGCLICLSHIKCTPKMKKFIPGRCHTYEGDKESAQGGIGEAIVDIPEIPGFKDLEPIEQFIAQVDLCVDCTTGCLKGLANVQCSDLLKKWLPQRCATFASKIQGQVDKIKGAGGD

SEQ ID NO: 16 - protein, 4 aa, Homo sapiens
  MISC_FEATURE (1)..(4): cleavage site for Furin
RRKR

SEQ ID NO: 17 - protein, 4 aa, Artificial
  substituted cleavage site for Furin
  MISC_FEATURE (1)..(4): Substituted cleavage site for Furin
GSGS

(19) United States
(12) Patent Application Publication
(10) Pub. No.: US 2023/0235017 A1
(43) Pub. Date: Jul. 27, 2023

(54) A DRUG SCREENING PLATFORM FOR ENDOSOMAL ENHANCERS TO TREAT ALZHEIMER'S DISEASE

(71) Applicant: Aarhus Universitet, Aarhus C (DK)

(72) Inventor: Olav Michael Andersen, Skamby (DK)

(21) Appl. No.: 18/001,510

(22) PCT Filed: Jun. 15, 2021
(86) PCT No.: PCT/EP2021/066116

§ 371 Date: Dec. 12, 2022

(30) Foreign Application Priority Data
Jun. 16, 2020 (EP) 20180166.9

(51) Int. Cl. C07K 14/705; C12N 9/16; G01N 33/50; C12N 15/62

(52) U.S. Cl. CPC C07K 14/70571; C12N 9/16; C12N 15/62; C12Y 301/03001; G01N 33/5076; C07K 2319/60; C07K 2319/61; G01N 2800/2821

(57) ABSTRACT

The present invention relates to a sorLA-based drug screening platform for use in screening compound libraries for an effect on endosomal activity.

### TECHNICAL FIELD

The present invention relates to a sorLA-based drug screening platform for use in screening small-compound libraries for an effect on endosomal activity. Compounds identified in the assay can be used in the treatment of Alzheimer’s disease (AD).

### BACKGROUND

Alzheimer’s disease (AD) is an incurable neurodegenerative disorder which affects millions of people worldwide. Its pathologies include Amyloid plaques and neurofibrillary tangles containing Amyloid b-peptide, also referred to Amyloid beta peptides or Abeta peptid, and tau, respectively. Several recent publications suggest endosomal dysfunction to be an upstream pathology that can lead to increased Amyloid and tau secretion from neurons (Small 2017, Small 2020), in line with endosomal dysfunction as one of the earliest and most well-studied cytopathologies of brains from AD patients. However, it is challenging to understand how the endosomal sorting system can be influenced, for example activated, to counteract endosomal dysfunction in AD. Consequently, there is a large unmet need to identify novel compounds which can be used as endosomal enhancers and thus be used in the treatment of AD. Likewise, there is a large unmet need to evaluate existing and future compounds for their potential negative side effect on the endosomal sorting system, for example compounds which would function as endosomal repressors and which may be detrimental to be used in a treatment, both for the general population and especially for AD patients which would be negatively affected by an additional inhibition of the endosomal sorting system.

Consequently, to facilitate the development of novel treatment options for AD, such as drug compounds, and to assess the safety of these treatment options for AD, technologies to assess their effect on the endosomal sorting system are needed.

### SUMMARY

SORL1 is a gene encoding an endosomal sorting receptor (sorLA), and is one of the main risk genes associated with the development of AD, for example sporadic/late-onset AD. The inventors of the present invention have realized the suitability of sorLA to assess the status and functionality of the endosomal sorting system, for example in the context of AD, and to assess if the endosomal system is in a normal state, in an activated state (higher activity than the normal state) or in an inhibited state (lower activity than the normal state). This is due to the realization of the inventors of the present invention that sorLA shed from the cell membrane can be used as a measure to assess the endosomal sorting system. SorLA (encoded by SORL1) is a neuronal receptor, which determines trafficking and processing of the amyloid precursor protein (APP). SorLA sorts through the secretory pathway to the cell surface and is internalized to early/sorting endosomes, where it recycles back to the plasma membrane or undergoes retrograde transport back to Golgi and/or the trans golgi network (TGN). It also undergoes ectodomain shedding and a soluble form of sorLA is found in the cerebrospinal fluid as well as in the culture medium of expressing cells.

The inventors have found that retrograde transport from the endosome affects complex-type N-glycosylation, which has been found to be a prerequisite for shedding of sorLA. The inventors have realized that shedding of sorLA can be used as a measure for endosomal activity. Increased shedding of sorLA reflects increased endosomal activity, while decreased shedding of sorLA reflects decreased endosomal activity. Conclusively, shedding of sorLA can be used as a measure on how a drug compound affects the endosomal sorting systems, for example acting as an endosomal activator or an endosomal repressor.

The present invention discloses a protein comprising sorLA fused with a reporter (“sorLA-reporter fragment”), for example secreted alkaline phosphatase (SEAP) or forms of Gaussia luciferase (Gluc). This fusion protein can be expressed in cells, and these cells can be used for screening small-compound libraries for compounds that can increase or decrease the endosomal activity. An increase in reporter activity, for example SEAP activity or Gluc activity, will in this setting represent an increase in shedding of sorLA, which represents an increase in the endosomal activity. A decrease in reporter activity, for example SEAP activity or Gluc activity, will in this setting represent a decrease in shedding of sorLA, which represents a decrease in the endosomal activity.

A compound, R55, has been shown in the literature to have an effect on endosomal activity and thereby a potential effect in the treatment of AD (Mecozzi 2014). It is the inventor’s understanding that R55 can be used as a proof of concept of the present invention.

The present invention describes a method for classifying compounds as either endosomal activity enhancing or endosomal activity repressing depending on their ability to either increase or decrease the amount of shed sorLA in the cell media of cells expressing a sorLA-reporter fusion protein. The disclosure further relates to said sorLA-reporter fusion protein, a polynucleotide encoding said fusion protein and a vector and a cell for expressing the fusion protein.

In one aspect, the present invention relates to a polynucleotide construct comprising
(a) a first polynucleotide encoding sorLA protein comprising or consisting of the amino acid sequence of SEQ ID NO: 10, or a sequence variant thereof, wherein the variant is at least 80 % identical to SEQ ID NO: 10, such as at least 95%, such as at least 99%; and
(b) a second polynucleotide encoding a reporter fragment,
wherein the first and the second polynucleotides are operably linked.

In a further aspect, the present invention is directed to a vector comprising the above described polynucleotide.

In a further aspect, the present invention is directed to a fusion protein encoded by above described polynucleotide or by the above described vector.

In a further aspect, the present invention is directed to a cell comprising the above described polynucleotide construct and/or the above described vector and/or the above described fusion protein.

In another aspect, the present invention is directed to a method for classifying a compound as endosomal enhancing or endosomal repressing, the method comprising the steps of:
(a) Providing above described cells, wherein the cells express a above described fusion protein consisting or comprising of a sorLA-reporter fragment;
(b) Contacting a first aliquot of cells of (a) with medium comprising a compound, and contacting a second aliquot of cells of (a) with medium not comprising a compound,
(c) Determining and comparing the activity of the sorLA-reporter fragment in the first and second aliquot, and
(d) Classifying the compound as
   i. endosomal enhancing if an increase in reporter fragment activity is observed in the first aliquot of cells compared to the second aliquot of cells, or
   ii. endosomal repressing if a decrease in reporter fragment activity is observed in the first aliquot of cells compared to the second aliquot of cells.

In another aspect, the present invention is directed to a method for classifying a sorLA mutation as endosomal enhancing or endosomal repressing, the method comprising the steps of:
(a) Providing above described cells, wherein the cells express above described fusion protein consisting or comprising of a sorLA-reporter fragment, and wherein the sorLA protein is 100% identical to the amino acid sequence of SEQ ID NO: 10, and
(b) Providing above described cells, wherein the cells express above described fusion protein consisting or comprising of a sorLA-reporter fragment, and wherein the sorLA protein is a variant comprising a mutation prevalent in a patient suffering from Alzheimer’s disease,
(c) Culturing the cells of (a) and (b)
(d) Determining and comparing the activity of the sorLA-reporter fragment in medium from cells of (a) and (b), and
(e) Classifying the sorLA mutation as
   i. endosomal enhancing if an increase in reporter fragment activity is observed in the medium of the cells of (b) compared to the medium of the cells of (a), or
   ii. endosomal repressing if a decrease in reporter fragment activity is observed in the medium of the cells of (b) compared to the medium of the cells of (a).

In another aspect, the present invention is directed to a method for testing if a patient identified with a SORL1 mutation will benefit from treatment with a compound, the method comprising the steps of:
(a) Providing above described cells, wherein the cells express above described fusion protein consisting or comprising of a sorLA-reporter fragment, and wherein the sorLA protein is a variant comprising said SORL1 mutation,
(b) Contacting a first aliquot of cells of (a) with medium comprising a compound, and contacting a second aliquot of cells of (a) with medium not comprising a compound,
(c) Determining and comparing the activity of the sorLA-reporter fragment in the first and second aliquot, and
(d) Concluding that the patient will
   i. benefit from treatment with the compound if an increase in reporter fragment activity is observed in the first aliquot of cells compared to the second aliquot of cells, or
   ii. not benefit from treatment with the compound if a decrease in reporter fragment activity is observed in the first aliquot of cells compared to the second aliquot of cells.

In another aspect, the present invention is directed to a kit for conducting a screening assay for classifying a compound as endosomal enhancing or endosomal repressing, the kit comprising
the above described polynucleotide construct, and/or
the above described vector, and/or
the above described cell;
and instructions for use.

### DESCRIPTION OF DRAWINGS

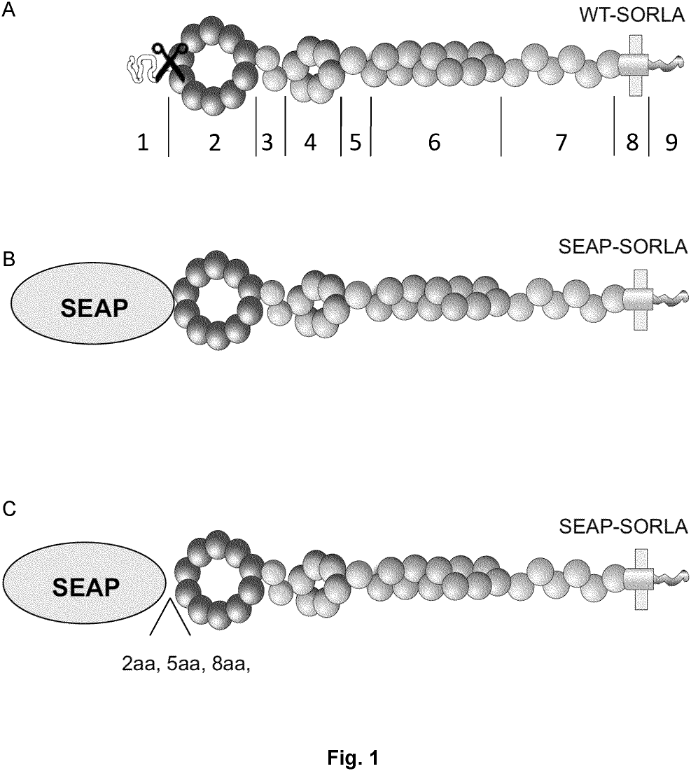

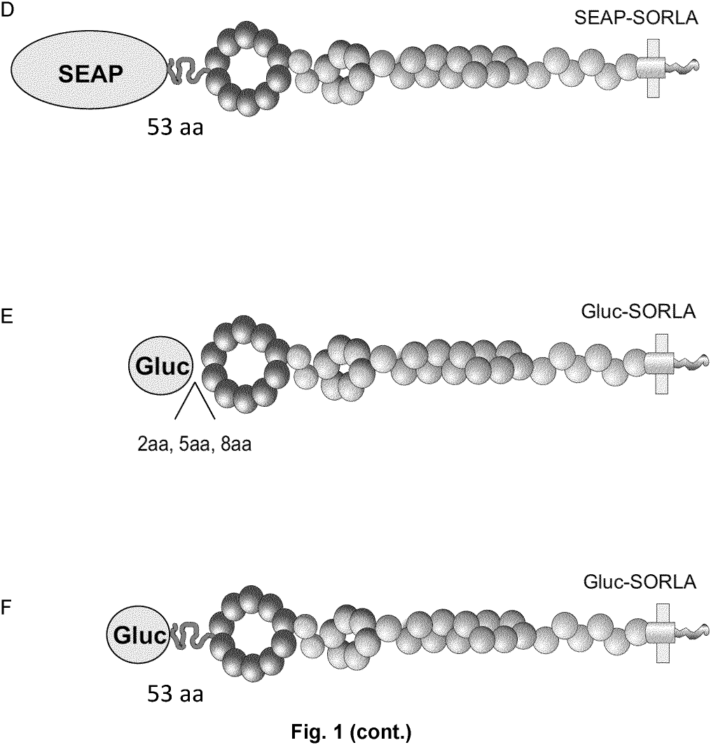

### FIG. 1: Schematic Presentation of WT-SORLA and Different Reporter Constructs

A) Schematic presentation of WT-sorLA (A). Numbers indicate sorLA domains: 1 = signal peptide and/or pro-peptide, 2 = VPS 10p-β-propeller domain, 3 = 10CC domain, 4 = YWTD-β-propeller domain, 5 = EGF domain, 6 = CR-cluster domain, 7 = 3Fn-cluster domain, 8 = transmembrane domain, 9 = tail domain. The scissor-symbol represents where the signal peptide and/or pro-peptide domain is substituted by N-terminal addition of a reporter molecule (as in B to F).

B) SEAP-sorLA where SEAP is added N-terminally substituting the pro-peptide.

C) SEAP-sorLA where SEAP and a linker of 2, 5 or 8 amino acids is added N-terminally substituting the pro-peptide.

D) SEAP-sorLA where SEAP and a linker of 53 amino acids is added N-terminally substituting the pro-peptide.

E) Gluc-sorLA where Gaussia Luciferase (Gluc) and a linker of 2, 5 or 8 amino acids is added N-terminally substituting the pro-peptide.

F) Gluc-sorLA where Gluc and a linker of 53 amino acids is added N-terminally substituting the pro-peptide.

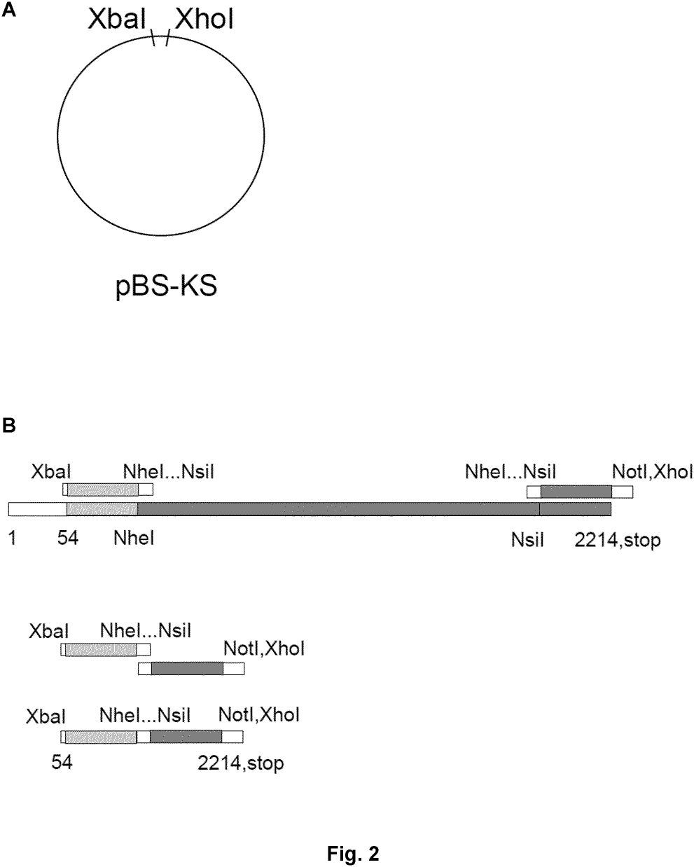

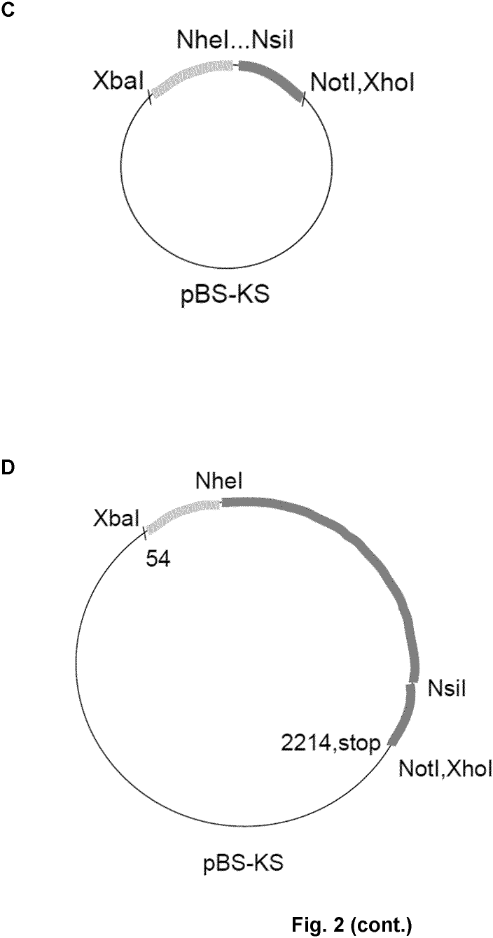

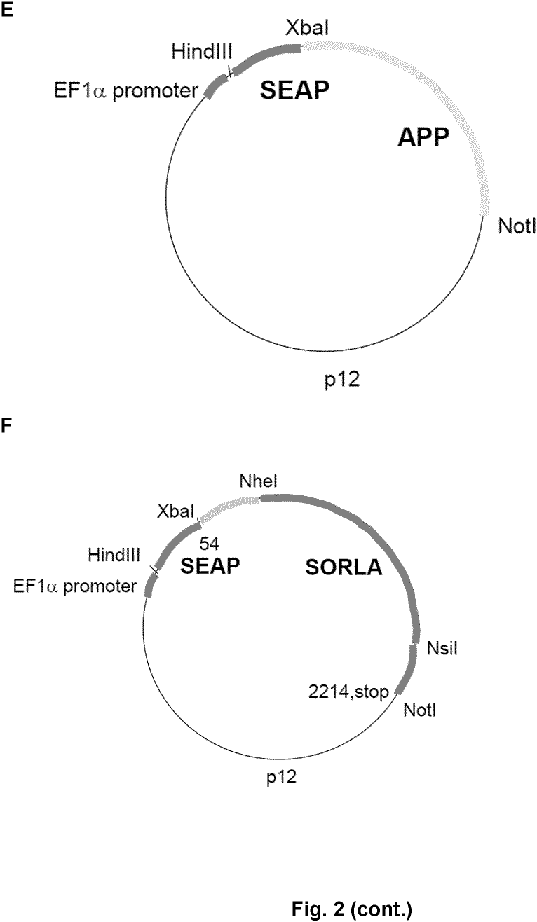

### FIG. 2: Cloning Strategy

Schematic overview of construction of a polynucleotide encoding SEAP-sorLA by PCR. A and B) Two fragments spanning residue 54 to the internal Nhel site and from internal Nsil site to residue 2214, stop codon of the sorLA cDNA were amplified by simple PCR, and then used as template in a nested PCR reaction to amplify their “sum” and provide flanking Xbal and Notl,Xhol recognition sites.

C) The fragment was digested with Xbal and Xhol restriction enzymes, and subsequently ligated into the pBS-KS cloning vector that was digested with the same two enzymes.

D) The long internal Nhel-Nsil fragment of the sorLA cDNA was isolated and ligated into the pBK-KS plasmid containing the flanking sorLA sequences. The product now contained the coding region of sorLA spanning residues 54 to 2214,stop and flanked by Xbal and Notl,Xhol sites.

E) A plasmid (backbone vector p12) containing SEAP fused to APP was a gift from Stefan Lichtenthaler, and where reading frame for APP insert can be replaced by Xbal (in-frame) and Notl fragments. This construct express SEAP-APP under control of EF1α promoter.

F) The sorLA fragment spanning residues 54-2214,stop was isolated after Xbal and Notl digest, and finally ligated into the p12 vector where the APP insert had been removed after Xbal and Notl digestion.

The final polynucleotide construct now contains sorLA 54-2214,stop in-frame with the SEAP in p12 plasmid and expressed under regulation of the EF1α promoter.

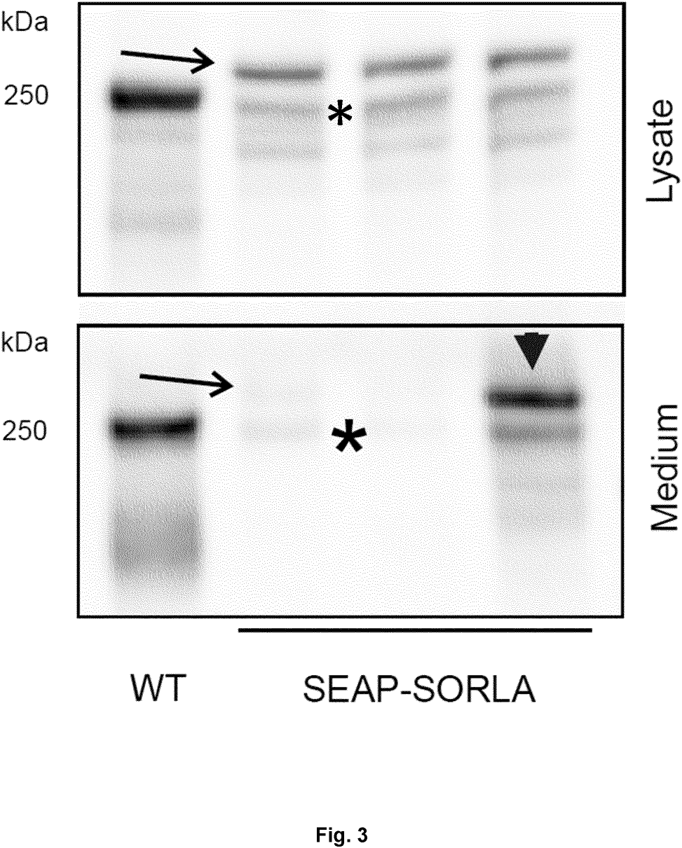

### FIG. 3: Western Blot Analysis

Western blot analysis of lysates (upper part of the figure): Western blot analysis of lysates from cells transfected with constructs for expression of WT-sorLA (lane 1) or SEAP-sorLA (lanes 2-4) confirmed expression of both proteins (top). Migration of the fusion protein (arrow) was slower than the WT protein (asterisk *). This correlates well with the longer polypeptide of the fusion protein compared to WT. Generally, lower expression of SEAP-sorLA compared to WT-sorLA in the lysates can be observed, due to a less active promoter in the SEAP-sorLA construct aiming for expression level more comparable to the endogenous situation.

Western blot analysis of medium (bottom part of the figure): The presence of shed SEAP-sorLA in the medium shows that the fusion protein does get cleaved and shed, likely in a ADAM17-dependent process similar to WT-sorLA. Loading of different levels of medium illustrates how cells grown in the presence of an endosomal enhancer (arrowhead) would be expected to lead to increased production of shed SEAP-sorLA.

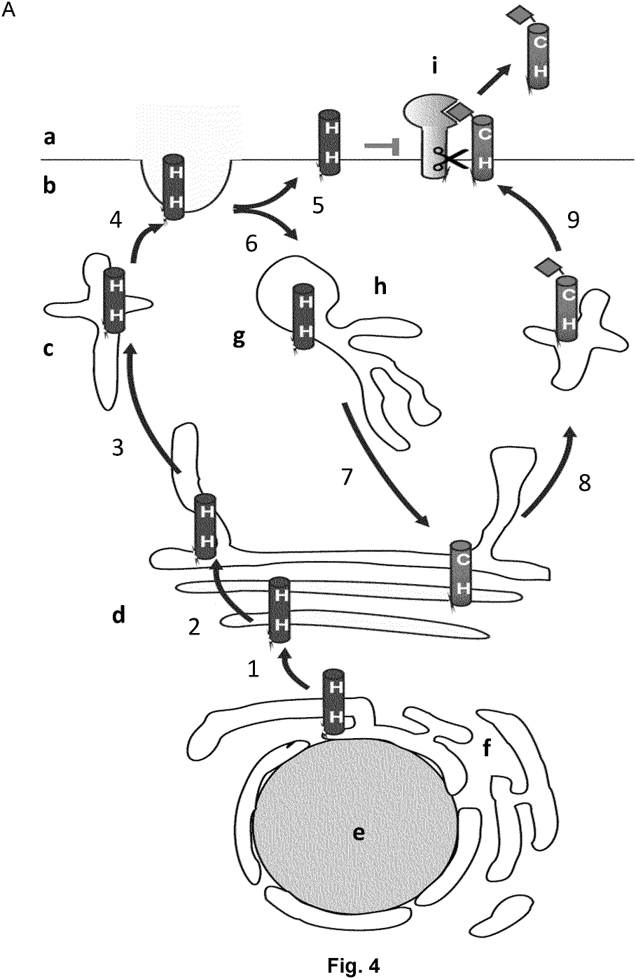

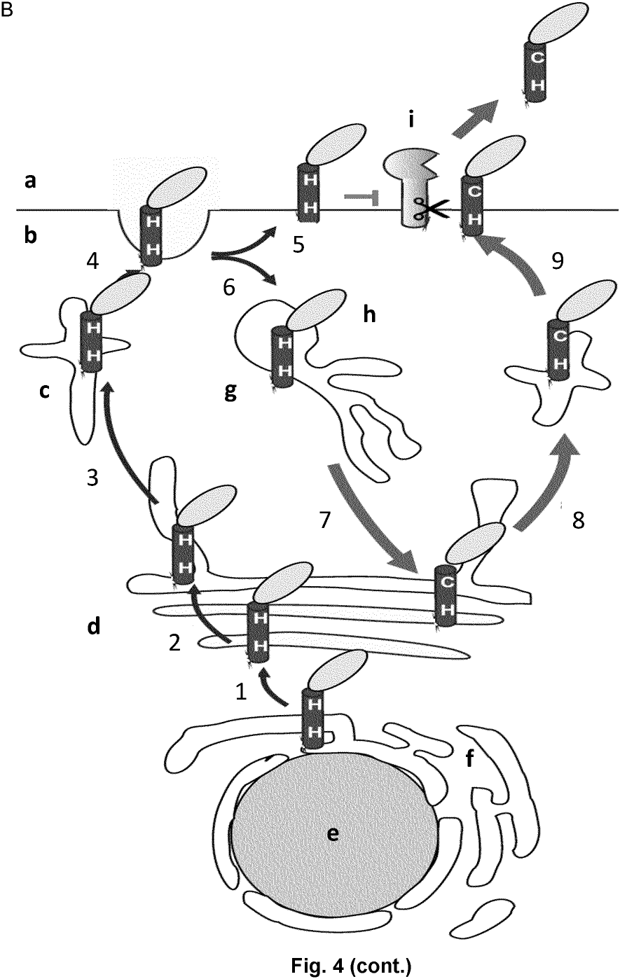

### FIG. 4: Schematic Presentation of Trafficking Pathways

A) Schematic presentation of sorLA trafficking pathways and maturation.

B) The panel illustrates how the SEAP-sorLA follows a similar trafficking pathway, and in cells grown in the presence of endosomal enhancers of, for example, the sorting itinerary steps (7-8-9), trafficking will be boosted and lead to increased production of shed SEAP-sorLA.

a: Extracellular space, b: Cytosol, c: trans golgi network (TGN), d: Golgi, e: Nucleus, f: endoplasmatic reticulum (ER), g: early endosome, h: tubular endosomal network (TEN), i: TNFα Converting Enzyme) TACE.

Numbers represent a model for sorLA trafficking, N-glycosylation and shedding. Newly synthesized sorLA undergoes glycosylation in the ER and after transfer to the cis-Golgi (1), immature sorLA is able to escape maturation of high-mannose (white H in the sorLA representation) N-glycans (2), and pass to the cell surface (3, 4). SorLA with the immature N-glycans is not substrate for TACE (5), but can internalize via clathrin-coated pits (6) to early endosomes from where it undergoes retrograde trafficking in a retromer-dependent route to the Golgi (7). Here N-glycans can mature to complex-type (white C in the sorLA representation) that return to the cell surface (8, 9) and mature sorLA is then accessible to TACE cleavage and shedding.

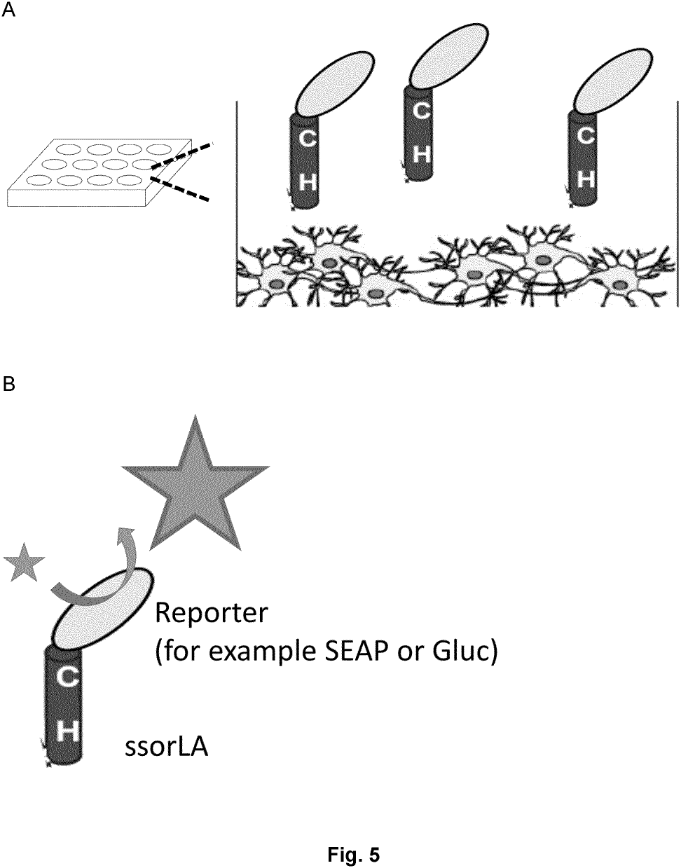

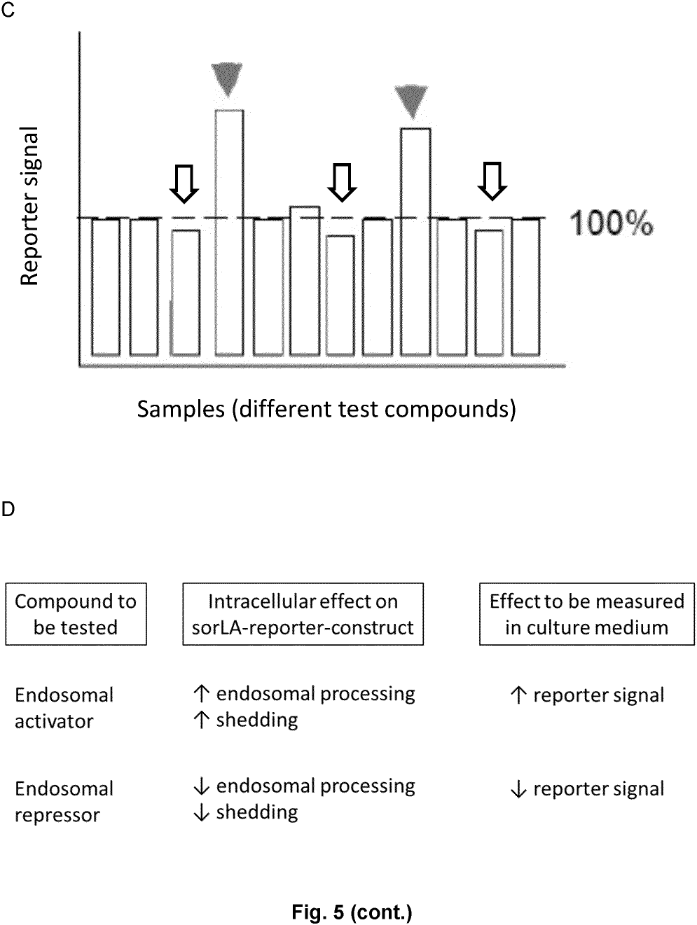

### FIG. 5: Assay

An assay to screen displayed libraries of compounds, siRNA or cDNA, using cells that express SEAP-sorLA grown in 96/384 well formats (A). Enzymatic activity in medium from cells will be quantified (B, C), and enhancers (grey arrow heads) of endosomal activity will be identified where the relative activity is higher in medium from compound-treated cells compared to vehicle-treated cells (non-treated cells (C). Alternatively, repressors of endosomal activity will be identified where the relative activity in medium from compound -treated cells is lower than in medium from vehicle-treated cells (non-treated cells. In summary, an increased reporter signal, reflecting increased endosomal processing and sorLA shedding, reflects the action of an endosomal activator or enhancer (D). A decreased reporter signal, reflecting decreased endosomal processing and sorLA shedding, reflects the action of an endosomal repressor.

### DETAILED DESCRIPTION

### Definitions

As used herein, the singular forms “a”, “an” and “the” include plural referents unless the context clearly states otherwise.

The term “some embodiments” can include one, or more than one embodiment.

The use of the word “a” or “an” when used throughout the text or in conjunction with the term “comprising” in the claims and/or the specification may mean “one,” but it is also consistent with the meaning of “one or more,” “at least one,” and “one or more than one.” Thus, for example, reference to “sorLA receptor construct” includes a plurality of such sorLA receptor constructs, such as one or more sorLA receptor constructs, at least one sorLA receptor construct, or two or more sorLA receptor constructs.

The term “sorLA” as used herein is synonymous to the terms SORLA, Sortilin-related receptor, sortilin related receptor 1, Low-density lipoprotein receptor relative with 11 ligand-binding repeats, LDLR relative with 11 ligand-binding repeats, LR11, SorLA-1, Sorting protein-related receptor containing LDLR class A repeats and gp250. Human sorLA is annotated in UniProt under the accession number Q92673.

The term “wildtype” as used herein and abbreviated “wt” is understood to describe a naturally occurring protein, i.e. naturally occurring sorLA.

The term “compound” as used herein includes any compound being tested or classified in the methods described herein. This includes for example, without limitation, small molecules, a peptides, polypeptides, macromolecules, a lipids, siRNAs, microRNAs and cDNAs. Compounds tested in the assay may in principle be any chemical compound, such as a naturally occurring compound, or a chemically synthesized compound that is identical or similar to a naturally occurring compound, or any chemically synthesized compound that does not occur in nature. A naturally occurring compound may be a compound that can be detected in or isolated from a multicellular or single cell organism, in particular a compound that can be detected in or isolated from an animal, a plant, a fungus, a yeast, bacterium, or any other cell-containing organism or in a virus. A chemically synthesized compound that does not occur in nature may be synthesized by combinatorial chemistry.

The term “reporter fragment” as used herein is synonymous to the term “reporter” and refers to a moiety that can be used to measure, and preferably quantify, a cellular process. Within the meaning of the present disclosure, the reporter fragments, fused to sorLA protein, is able to “report”, meaning indicate, sorLA which is shed from the cell surface into the medium, and consequently shed sorLA (ssorLA) can be measured and quantified in the medium. A suitable reporter allows for sensitive, accurate and preferably quantifyable measurement. The present invention focuses on the usage of reporter enzymes, such as Gaussia luciferase (Gluc) or secreted alkaline phosphatase (SEAP). The activity of these reporter enzymes can be measured, and consequently the activity of the reporter can be related to the amount of the sorLA-reporter fusion protein which is shed into the medium. As the inventors of the present invention have realized, shed sorLA is a measure of endosomal processing/functionality. By measuring or determining the “activity of the reporter fragment” it is meant that the reaction of a substrate by its respective reporter enzyme is measured or determined. Upon incubation with their respective substrate, the reporter enzyme catalyzes a measureable reaction, e.g. measurable by light emission or colorimetric changes. For example, luciferases such as Gaussia luciferase catalyze light emission in the presence of their substrates (“luciferins”) which can be measured.

For high-throughput assays, for which the herein disclosed polynucleotide construct, vector, fusion protein, cell or methods can be used, optimal reporter enzymes display one or ideally more of the following properties: high light output for sensitivity, non-invasive monitoring of enzymatic activity, enzyme stability over various conditions, catalysis of stable light emission for minimal variability between a multitude of screened samples.

As another example, alkaline phosphatases such as SEAP catalyze a colorimetric changes in the presence of their substrates which can be measured.

The term “sorLA-reporter fragment” as used herein refers to a fusion protein comprising or consisting of a reporter fragment as disclosed herein, being fused to the protein sorLA, either directly or via a linker as disclosed herein.

### Polynucleotide Construct

In one aspect, the present invention relates to a polynucleotide construct comprising
(a) a first polynucleotide encoding sorLA protein comprising or consisting of the amino acid sequence of SEQ ID NO: 10, or a sequence variant thereof, wherein the variant is at least 80 % identical to SEQ ID NO: 10, such as at least 95%, such as at least 99%; and
(b) a second polynucleotide encoding a reporter fragment,
wherein the first and the second polynucleotides are operably linked.

In some embodiments of the present disclosure, the sorLA protein is 100% identical to the amino acid sequence of SEQ ID NO: 10.

In some embodiments of the present disclosure, said sorLA variant comprises a mutation prevalent in a patient suffering from Alzheimer’s disease.

The person skilled in the art will know how to identify said mutation prevalent in Alzheimer’s disease. Certain mutations are known to date, and other mutations will be identified in the future, for example through genome-wide studies.

In some embodiments of the present disclosure, said mutation is a missense mutation.

### Reporter

In some embodiments of the present disclosure, the reporter fragment is selected from the group consisting of Gaussia luciferase (Gluc), enhanced Gaussia luciferase (eGluc) secreted alkaline phosphatase (SEAP), firefly (Photinus pyralis) luciferase and Renilla reniformis luciferase; or optimized variants of these.

In some embodiments of the present disclosure, the reporter fragment is Gaussia luciferase (Gluc), or an optimized variant thereof.

In some embodiments of the present disclosure, the reporter fragment is enhanced Gaussia luciferase (eGluc).

In some embodiments of the present disclosure, the reporter fragment is 100% identical to the amino acid sequence of SEQ ID NO: 15.

In some embodiments of the present disclosure, the reporter fragment is selected from Gluc-M431I, Gluc-M110I and GlucM43I.

In some embodiments of the present disclosure, the reporter fragment is secreted alkaline phosphatase (SEAP), or an optimized variant thereof.

In some embodiments of the present disclosure, the reporter fragment is 100% identical to the amino acid sequence of SEQ ID NO: 11.

In some embodiments of the present disclosure, upon expression the reporter fragment is fused with sorLA at the N-terminal of sorLA.

In one embodiment the reporter fragment is fused at any position in the sorLA construct

In preferred embodiments the reporter fragment is fused with sorLA at the N-terminal.

Using a fusion protein where sorLA is fused to a reporter fragment, allows for easy quantification of ssorLA. Examples of reporter fragments are alkaline phosphatase or luciferase. Such a sorLA-reporter fusion protein, is ideal for setting up high-throughput screenings protocols to identify compounds that influence endosomal activity. Alkaline phosphatase and luciferase reporter constructs are highly sensitive, they display a wide dynamic range and a high saturation threshold. Finally, they require very little hands-on-time. All these parameters allows for setting up a high-throughput screening.

The invention also relates to a polynucleotide encoding the fusion protein as described above. The polynucleotide may comprise a DNA nucleotide sequence or an RNA nucleotide sequence, such as genomic DNA, cDNA, and RNA sequences, either double stranded or single stranded.

Thus, in one embodiment the invention relates to an isolated polynucleotide, encoding the sorLA-reporter fusion protein

### Linker

In some embodiments of the present disclosure, above described polynucleotide construct is further comprising a third polynucleotide encoding a linker, wherein the first, the second and the polynucleotides are operably linked.

The person skilled in the art will appreciate that a linker may be used to enhance or ensure functionality of a fusion protein. Some fusion proteins are functional with the addition of a linker linking elements of the fusion protein. Some fusion proteins need the addition of a linker linking elements of the fusion protein.

In some embodiments of the present disclosure, the linker ensures solubility and/or proper folding and/or functional 3D-confirmation of the expression product.

In some embodiments of the present disclosure, the linker links the sorLA protein to the reporter fragment.

In some embodiments of the present disclosure, upon expression the linker is fused with sorLA at the N-terminal of sorLA.

In some embodiments of the present disclosure, upon expression the reporter fragment is fused with the linker at the N-terminal of the linker.

In some embodiments of the present disclosure, the linker consists of at least one amino acid, for example 2 amino acids, such as 3 amino acids, for example 4 amino acids, such as 5 amino acids, for example 6 amino acids, such as 7 amino acids, for example 8 amino acids, such as 9 amino acids, for example 19 amino acids, such as 11 amino acids, for example 12 amino acids, such as 13 amino acids, for example 14 amino acids, such as 15 amino acids, for example 16 amino acids, such as 17 amino acids, for example 18 amino acids, such as 19 amino acids, for example 20 amino acids, such as 25 amino acids, for example 30 amino acids, such as 35 amino acids, for example 40 amino acids, such as 45 amino acids, for example 50 amino acids, such as 55 amino acids, for example 60 amino acids, such as 65 amino acids, for example 70 amino acids, such as 75 amino acids, for example 80 amino acids, such as 85 amino acids, for example 90 amino acids, such as 95 amino acids, for example 100 amino acids.

In some embodiments of the present disclosure, the linker consists of 2 amino acids.

In some embodiments of the present disclosure, the linker consists of 5 amino acids.

In some embodiments of the present disclosure, the linker consists of 8 amino acids.

In some embodiments of the present disclosure, the linker consists of 53 amino acids.

In some embodiments of the present disclosure, the linker consists of the of an amino acid sequence of LE.

In some embodiments of the present disclosure, the linker consists of the of an amino acid sequence of LSRSE (SEQ ID NO: 12).

In some embodiments of the present disclosure, the linker consists of the of an amino acid sequence of LSRSSGGE (SEQ ID NO: 13).

In some embodiments of the present disclosure, the linker consists of the of an amino acid sequence of

EVWTQRLHGGSAPLPQDRGFLVVQGDPRELRLWARGDARGASRADEKPLG
SGS(SEQ ID NO: 14).

In some embodiments of the present disclosure, above describe polynucleotide construct is encoding the fusion protein comprising or consisting of an amino acid sequence of SEQ ID NO: 6.

In some embodiments of the present disclosure, above describe polynucleotide construct is encoding a fusion protein comprising or consisting of an amino acid sequence of SEQ ID NO: 7.

In some embodiments of the present disclosure, above describe polynucleotide construct is encoding a fusion protein comprising or consisting of an amino acid sequence of SEQ ID NO: 8.

In some embodiments of the present disclosure, above describe polynucleotide construct is encoding a fusion protein comprising or consisting of an amino acid sequence of SEQ ID NO: 9.

In some embodiments of the present disclosure, above describe polynucleotide construct is encoding a fusion protein comprising or consisting of an amino acid sequence of SEQ ID NO: 1.

In some embodiments of the present disclosure, above describe polynucleotide construct is encoding a fusion protein comprising or consisting of an amino acid sequence of SEQ ID NO: 2.

In some embodiments of the present disclosure, above describe polynucleotide construct is encoding a fusion protein comprising or consisting of an amino acid sequence of SEQ ID NO: 3.

In some embodiments of the present disclosure, above describe polynucleotide construct is encoding a fusion protein comprising or consisting of an amino acid sequence of SEQ ID NO: 4.

In some embodiments of the present disclosure, above describe polynucleotide construct is encoding a fusion protein comprising or consisting of an amino acid sequence of SEQ ID NO: 5.

### Vector

In a further aspect, the present invention is directed to a vector comprising the above described polynucleotide.

In some embodiments of the present disclosure, the vector is selected from the group consisting of plasmids, viral vectors, and cosmids.

In some embodiments of the present disclosure, the vector is a plasmid vector.

In some embodiments of the present disclosure, the vector is a viral vector.

In some embodiments of the present disclosure, the vector is functional in mammalian cells.

In some embodiments of the present disclosure, vector further comprises 5′ and 3′ terminal repeats.

In some embodiments of the present disclosure, the vector comprises a promoter.

In some embodiments of the present disclosure, said promoter is specific for mammalian cells.

In some embodiments of the present disclosure, said mammalian cell is a neural cell.

In some embodiments of the present disclosure, the vector further comprises a polyadenylation sequence.

In some embodiments of the present disclosure, said nucleotide sequence is operably linked to a post-transcriptional regulatory element.

Another aspect of the invention relates to a construct or a vector comprising the isolated polynucleotide as described above.

The vector is for transfecting a host cell and expression of the fusion protein encoded by the polynucleotide described above, i.e. an expression vector.

### Fusion Protein

In a further aspect, the present invention is directed to a fusion protein encoded by above described polynucleotide or by the above described vector.

In some embodiments of the present disclosure, the fusion protein comprises or consists of an amino acid sequence of SEQ ID NO: 6.

In some embodiments of the present disclosure, the fusion protein comprises or consists of an amino acid sequence of SEQ ID NO: 7.

In some embodiments of the present disclosure, the fusion protein comprises or consists of an amino acid sequence of SEQ ID NO: 8.

In some embodiments of the present disclosure, the fusion protein comprises or consists of an amino acid sequence of SEQ ID NO: 9.

In some embodiments of the present disclosure, the fusion protein comprises or consists of an amino acid sequence of SEQ ID NO: 1.

In some embodiments of the present disclosure, the fusion protein comprises or consists of an amino acid sequence of SEQ ID NO: 2.

In some embodiments of the present disclosure, the fusion protein comprises or consists of an amino acid sequence of SEQ ID NO: 3.

In some embodiments of the present disclosure, the fusion protein comprises or consists of an amino acid sequence of SEQ ID NO: 4.

In some embodiments of the present disclosure, the fusion protein comprises or consists of an amino acid sequence of SEQ ID NO: 5.

Such a sorLA-reporter fusion protein, is ideal for setting up high-throughput screenings. Furthermore, a fusion protein comprising sorLA and a reporter fragment could be used to further elucidate molecular mechanisms underlying the link between abnormal endosomal activity and neurodegenerative diseases.

The person skilled in the art will appreciate that a fusion protein can be understood as a polypeptide.

### Cell

In a further aspect, the present invention is directed to a cell comprising the above described polynucleotide construct and/or the above described vector and/or the above described fusion protein.

In some embodiments of the present disclosure, said cell is of mammalian origin.

In some embodiments of the present disclosure, said cell is of human origin.

In some embodiments of the present disclosure, said cell is selected from the group consisting of HeLa, HEK and SH-SY5Y cells.

In some embodiments of the present disclosure, said cell is a SH-SY5Y cell.

### Methods to Assess a Compound

In another aspect, the present invention is directed to a method for classifying a compound as endosomal enhancing or endosomal repressing, the method comprising the steps of:
(a) Providing above described cells, wherein the cells express a above described fusion protein consisting or comprising of a sorLA-reporter fragment;
(b) Contacting a first aliquot of cells of (a) with medium comprising a compound, and contacting a second aliquot of cells of (a) with medium not comprising a compound,
(c) Determining and comparing the activity of the sorLA-reporter fragment in the first and second aliquot, and
(d) Classifying the compound as
   i. endosomal enhancing if an increase in reporter fragment activity is observed in the first aliquot of cells compared to the second aliquot of cells, or
   ii. endosomal repressing if a decrease in reporter fragment activity is observed in the first aliquot of cells compared to the second aliquot of cells.

The person skilled in the art will be able to select suitable media for the cells employed in the methods disclosed herein.

In some embodiments of the present disclosure, said method is used to identify compounds that increase the amount of sorLA-reporter fusion protein in the cell media.

In some embodiments of the present disclosure, said method is used to identify compounds that decrease the amount of sorLA-reporter fusion protein in the cell media.

In some embodiments of the present disclosure, said classified compound
suitable for use in the treatment of Alzheimer’s disease (AD) if the compound is classified as endosomal enhancing, or
unsuitable for use in the treatment of Alzheimer’s disease (AD) if the compound is classified as endosomal repressing.

The person skilled in the art will appreciate that a compound suitable for use in the treatment of AD enhances endosomal processing, and as such is able to counteract endosomal deficiencies an AD patient may have. Compound unsuitable for use in the treatment of AD would not have enhance endosomal processing. Alternatively, a compound unsuitable for use in the treatment of AD would decrease endosomal processing, acting as an endosomal repressor, and would as such aggravate the endosomal deficiencies an AD patient may have.

### Methods to Assess SORL1 Mutations

In another aspect, the present invention is directed to a method for classifying a sorLA mutation as endosomal enhancing or endosomal repressing, the method comprising the steps of:
(a) Providing above described cells, wherein the cells express above described fusion protein consisting or comprising of a sorLA-reporter fragment,
   and wherein the sorLA protein is 100% identical to the amino acid sequence of SEQ ID NO: 10, and
(b) Providing above described cells, wherein the cells express above described fusion protein consisting or comprising of a sorLA-reporter fragment,
   and wherein the sorLA protein is a variant comprising a mutation prevalent in a patient suffering from Alzheimer’s disease,
(c) Culturing the cells of (a) and (b)
(d) Determining and comparing the activity of the sorLA-reporter fragment in medium from cells of (a) and (b), and
(e) Classifying the sorLA mutation as
   i. endosomal enhancing if an increase in reporter fragment activity is observed in the medium of the cells of (b) compared to the medium of the cells of (a), or
   ii. endosomal repressing if a decrease in reporter fragment activity is observed in the medium of the cells of (b) compared to the medium of the cells of (a).

In another aspect, the present invention is directed to a method for testing if a patient identified with a SORL1 mutation will benefit from treatment with a compound, the method comprising the steps of:
(a) Providing above described cells, wherein the cells express above described fusion protein consisting or comprising of a sorLA-reporter fragment,
   and wherein the sorLA protein is a variant comprising said SORL1 mutation,
(b) Contacting a first aliquot of cells of (a) with medium comprising a compound, and contacting a second aliquot of cells of (a) with medium not comprising a compound,
(c) Determining and comparing the activity of the sorLA-reporter fragment in the first and second aliquot, and
(d) Concluding that the patient will
   i. benefit from treatment with the compound if an increase in reporter fragment activity is observed in the first aliquot of cells compared to the second aliquot of cells, or
   ii. not benefit from treatment with the compound if a decrease in reporter fragment activity is observed in the first aliquot of cells compared to the second aliquot of cells.

In some embodiments of the present disclosure, the sorLA-reporter fragment consists of a reporter fragment fused to the protein sorLA, either directly or via a linker as described above.

In some embodiments of the present disclosure, the sorLA-reporter fragment comprises a reporter fragment fused to the protein sorLA, either directly or via a linker as described above.

In some embodiments of the present disclosure, determining the activity of the substrate comprises the addition of a substrate for the reporter fragments.

In some embodiments of the present disclosure, the substrate for the reporter fragments is a substrate suitable for Gaussia luciferase (Gluc), enhanced Gaussia luciferase (eGluc) secreted alkaline phosphatase (SEAP), firefly (Photinus pyralis) luciferase and Renilla reniformis luciferase; or optimized variants of these.

In some embodiments of the present disclosure, determining the activity of the substrate comprises the addition of a substrate for the reporter fragments, and incubating with the substrate for a duration of time.

The person skilled in the art will be acquainted with standard procedures for the determination of reporter fragment activity, such as the enzymatic reporter fragments disclosed herein, and will be able to determine suitable incubation lengths and read-out parameters.

The person skilled in the art will understand that the herein disclosed methods can, for example, be used in an assay, for example a screening assay to identify compounds which have endosomal activating or endosomal repressing functions. The herein disclosed methods can, for example, be used as a drug screening platform for identifying endosomal enhancers to treat Alzheimer’s disease.

The herein disclosed methods, or assays employing these methods, utilize the finding, that SorLA is a neuronal sorting receptor implicated in sporadic forms of AD. It decreases the amyloidogenic cleavage of the Amyloid precursor protein. AD patients show reduced expression of sorLA in neurons and sorLA variants with inactivating mutations are associated with AD risk. Thus, soluble sorLA (ssorLA) levels in cerebrospinal fluid can serve as a biomarker for AD risk. If the levels of ssorLA are lower than in the general (healthy) population, a risk for established or developing AD can be stated. The studies underlying the herein disclosed invention further show how TACE-mediated shedding of sorLA from the cell membrane requires complex-type N-glycosylation, and the conversion of sorLA high-mannose glycoforms to complex-type glycoforms occurs only after transport to the cell membrane and during retrograde trafficking. Thereby, we have established that SorLA exists as two distinct N-glycoforms at the cell surface, exclusive high-mannose and partial complex-type, and only the complex-type glycoform is shed.

Examples 2 and 3 show, that a sorLA-reporter fragment construct displays wildtype functionality. Hereby, the extracellular activity of the reporter fragment can indicate the level of shed sorLA (ssorLA), which is the mature sorLA that has been to the cell surface and undergone endocytosis and retrograde sorting via the endosomal trafficking pathway, and subsequently shed from the cell surface by ADAM17-dependent proteolytic processing. Retromer-facilitated trafficking is essential for sorLA to perform a protective role in development of AD. This is in agreement with retromer dysfunction leading to core pathological features of AD. Factors that increase ssorLA production can therefore be expected to be potential drug candidates for treatment of AD.

In some embodiments of the present disclosure the method is used to identify compounds that increases the amount of sorLA-reporter fusion protein in the cell media. As an increase in sorLA-fusion protein in the cell media represents an increase in ssorLA, such compounds are expected to have an activating effect on endosomal activity. Compounds increasing the amount of sorLA-reporter fusion protein in the cell media are therefore expected to be potential drug candidates for treatment of AD.

Certain pathogenic variants of SORL1 are involved in the familiar/early-onset form of AD.

Furthermore, endosome dysfunction are among the earliest neuropathological features to develop and have now been closely linked to genetic risk factors for AD. It would thus be of key interest to identify compounds that could regulate endosomal dysfunction to prevent progression of AD at a very early stage.

### Compounds

The person skilled in the art will appreciate that the herein described polynucleotide construct, vector, fusion protein, cell or methods can be used to assess compounds, i.e. compounds to be tested for their properties of being endosomal enhancers or repressors. These compounds may be referred to as “test compounds”. The test compound may be any molecule which is analysed with regard to its properties of being an endosomal enhancer or an endosomal repressor. For example, the test compound may be a small molecule, a peptide, a polypeptide, a macromolecule or a lipid. The test compound may also be a molecule affecting components of the endosomal maturation and trafficking machinery on a genetic and/or gene regulatory and/or transcriptome level. The person skilled in the art will know suitable compounds, for example but not limited to compounds belonging to CRISPR/Cas9 arrayed libraries, siRNAs, micro RNAs or cDNAs.

The test compound is analysed for its interaction or effect on one or more components of the endosomal maturation and trafficking machinery which leads to maturation and/or shedding of sorLA, either in an activating or enhancing or stimulatory way (endosomal enhancer), or in an inhibiting or inactivating or repressing way (endosomal repressor).

In some embodiments of the present disclosure, the compound is selected from the group consisting of small molecules, a peptides, polypeptides, macromolecules, a lipids, siRNAs, microRNAs and cDNAs.

In some embodiments of the present disclosure, the compound is derived from CRISPR/Cas9 arrayed libraries.

### Kit

In another aspect, the present invention is directed to a kit for conducting a screening assay for classifying a compound as endosomal enhancing or endosomal repressing, the kit comprising
the above described polynucleotide construct, and/or
the above described vector, and/or
the above described cell;
and instructions for use.

### EXAMPLES

### Example 1: Construction of Polynucleotide Encoding SEAP-sorLA

### Aim

Construction of the SORLA reporter construct

### Material and Methods

FIG. 1 depicts a schematic presentation of WT-sorLA (FIG. 1A ) and SEAP-sorLA (FIGS. 1 B, C, D), where SEAP is added N-terminally substituting the pro-peptide. The following procedure describes the generation of a construct where SEAP was connected to sorLA via a 2 amino acids linker (leucine, glutamic acid), resulting in the construct with an amino acid sequence of SEQ ID NO: 1 and FIG. 1C.

Similar procedures may be used where SEAP is directly connected to sorLA (SEQ ID NO: 5 and FIG. 1B), or where linkers of different length are added (see Example 4).

The polynucleotide construct encoding the SEAP-sorLA protein was prepared by first using a two-step PCR for amplification of small sorLA insert according to the following procedure (see FIG. 2 for schematic illustration):

Two fragments spanning residue 54 to the internal Nhel site and from internal Nsil site to residue 2214+stop codon of the sorLA cDNA were amplified by simple PCR, and then used as template in a nested PCR reaction to amplify their “sum” and provide flanking Xbal and Notl,Xhol recognition sites (FIGS. 2 A-B).

This fragment was digested with Xbal and Xhol restriction enzymes, and subsequently ligated into the pBS-KS cloning vector that was digested with the same two enzymes (FIG. 2C).

The long internal Nhel-Nsil fragment of the sorLA cDNA was isolated and ligated into the pBK-KS plasmid containing the flanking sorLA sequences. The product now contained the coding region of sorLA spanning residues 54 to 2214,stop and flanked by Xbal and Notl,Xhol sites (FIG. 2D).

A plasmid (backbone vector p12) containing SEAP fused to APP was a gift from Stefan Lichtenthaler, and where reading frame for APP insert can be replaced by Xbal (in-frame) and Notl fragments. This construct expresses SEAP-APP under control of EF1α promoter (FIG. 2E).

The sorLA fragment spanning residues 54-2214,stop was isolated after Xbal and Notl digest, and finally ligated into the p12 vector where the APP insert had been removed after Xbal and Notl digestion.

The final polynucleotide construct now contains sorLA 54-2214,stop in-frame with the SEAP in p12 plasmid and expressed under regulation of the EF1α promoter (FIG. 2F).

### Results

SEAP-SORLA constructs were established.

### Conclusions

The established SEAP-SORLA constructs can be used to evaluate their functionality, and to further optimize them, for example as described in Example 4 by testing different variants of linkers between the reporter (e.g. SEAP) and SORLA or as described in Example 5 by using an alternative reporter (Gaussia luciferase reporter instead of SEAP).

### Example 2: Control Experiments

### Aim:

To evaluate the functionality of the SORLA-reporter construct.

### Material and Methods

### Positive and Negative Controls

The chemical chaperone R55 could be used to boost activity of retromer (thereby functioning as a “endosomal activator”) and thus enhance sorLA endosomal sorting, leading to increased shed sorLA (ssorLA) production and thus increased reporter activity in conditioned media. R55 can therefore be included in an assay as a positive control to validate the assay as a measure of endosomal activity, for example during testing and optimization of the SORLA-reporter construct. As such, the compound R55 can be used as a proof-of-concept and to validate the herein described assay.

It is also possible to include negative controls in the assay, for example agents which decrease sorLA endosomal sorting, leading to decreased shed sorLA (ssorLA) production and thus decreased reporter activity in conditioned media (thereby functioning as a “endosomal repressor” or “endosomal inactivator”).

Similarly, suitable negative controls could also be sorLA constructs, which are altered so that sorLA endosomal sorting is hampered, thus leading to decreased shed sorLA (ssorLA) production and thus decreased reporter activity in conditioned media.

Furthermore, and for example in relation to above detailed option, the sorLA fusion protein described above, which will be designed carrying a mutation of the sorLA tail FANSHY motif (Fjorback 2012) will show a strongly reduced activity and thus a decreased production of shed sorLA-reporter fragment construct. This will confirm that the sorLA-reporter fragment construct displays wildtype functionality. For example, it will be shown that introducing the mutation of the sorLA tail FANSHY motif will affect the reporter-sorLA construct in the same way as it would affect wt-sorLA.

Any identified compound could be tested for effect on sorLA-FANSHY as this is a negative control for the assay.

Further, confirming that the fusion protein relies (as the wt protein) on maturation of N-glycosylations acquired during endosomal trafficking (similar to WT-sorLA), cells treated with knockdown for retromer-components (VPS26 and VPS35) or MGAT1 will show a decrease in shed sorLA-reporter fragment production and enzymatic activity. Similarly, reporter activity in the medium will decrease for cells treated with the endocytosis inhibitors like Chlorpromazin (CPZ), dynasore (DYN) or pitstop2, since this will lead to a decrease of shed sorLA-reporter construct.

### Detection of the SEAP-SORLA Reporter Construct

Three batches of SEAP-SORLA-constructs were established as detailed in Example 1. Cells were transfected with the constructs. Medium (supernatant) was collected and the cells were subsequently lysed. Medium and lysate were prepared and analysed by Western Blotting (FIG. 3). The Western Blot was probed with an anti-SORLA antibody.

### Results

The Western Blot was probed with an anti-SORLA antibody, confirming expression of both WT-SORLA (top = lysate) and SEAP SORLA (bottom = medium). The arrow in FIG. 3 indicates the SEAP-SORLA construct which as expected runs higher in the gel due to the higher molecular weight compared to the bands indicating wt-SORLA (asterisk *). Generally, lower expression of SEAP-sorLA compared to WT-sorLA in the lysates can be observed, due to a less active promoter in the SEAP-sorLA construct aiming for expression level more comparable to the endogenous situation. For the lysate samples, the lanes indicated by SEAP-SORLA represent replicates. For the medium samples, the lanes indicated by SEAP-SORLA represent replicates, however the last lane to the right (lane 4) was loaded with more medium. In this lane, the SORLA signal is much stronger and this lane represents how an expected result would look if an endosomal activator would be used leading to increased shedding of a SORLA-reporter construct into the medium (arrowhead). Western blot analysis of medium (bottom part of the figure) confirmed the presence of shed SEAP-sorLA in the medium showing that the fusion protein got cleaved and shed, likely in a ADAM17-dependent process similar to WT-sorLA.

FIG. 4 shows a schematic presentation of SORLA trafficking pathways as proposed in Christensen et al, 2020. FIG. 4A illustrates that synthesized sorLA undergoes glycosylation in the ER and after transfer to the cis-Golgi (1), immature sorLA is able to escape maturation of high-mannose (H) N-glycans (2), and pass to the cell surface (3, 4). SorLA with the immature N-glycans is not substrate for TACE (5), but can internalize via clathrin-coated pits (6) to early endosomes from where it undergoes retrograde trafficking in a retromer-dependent route to the Golgi (7). Here N-glycans can mature to complex-type (C) that return to the cell surface (8, 9) and mature sorLA is then accessible to TACE cleavage and shedding. FIG. 4B illustrates how the SEAP-sorLA follows a similar trafficking pathway. This is confirmed by Western Blotting as detailed above and in FIG. 3. An antibody against SorLA identifies SEAP-SORLA in the medium, running slower in the gel than wt SORLA due to a higher molecular weight (higher band on gel/blot). Conclusively, SEAP-SORLA is shedded similar to wt-SORLA, and as such undergoes the same endosomal trafficking and modification as wt-SORLA. Any construct with another reporter fragment is expected to follow this trafficking pathway as well.

Conclusively, the sorLA trafficking pathway will be boosted in cells grown in the presence of endosomal enhancers of, for example, the sorting itinerary steps (7-8-9 in FIGS. 4 A or B ), leading to increased production of shed SEAP-sorLA.

Further, the sorLA trafficking pathway will be inhibited in cells grown in the presence of endosomal repressors or inactivators of, for example, the sorting itinerary steps (7-8-9 in FIGS. 4 A or B), leading to decreased production of shed SEAP-sorLA.

Our results indicate that the established SEAP-SORLA construct can be transfected into cells and detected in cell lysate and in medium. This also shows that SEAP-SORLA is functional and gets shed similar to wt-SORLA.

### Conclusion

The SEAP-SORLA construct can be used to test the effect of endosomal activators or endosomal repressors leading to increased shed SEAP-SORLA or decreased shed SEAP-SORLA, respectively. Based on this, the assay can be further validated using various positive and negative controls i.e. factors or SORLA mutations know to increase or decrease SORLA shedding.

### Example 3: Assay Protocol

### Aim:

Description of the assay.

### Material and Methods

A SH-SY5Y cell line stably expressing the SEAP-sorLA construct as described in Example 1 is generated. Other cell lines, such as other human cell lines, for example HeLa, HEK, SY5Y cell lines, could also be used.

Cells are seeded in 96/384 well plates and cultured until reaching a density of 80-100% confluence (FIG. 5A). To some cells test compounds are then added together with fresh medium while to other cells a vehicle and fresh medium is added. For example, in the case of adding a test compound, the compound might be dissolved in a vehicle such as a buffer (the “test sample”). In the case of not adding a test compound, only the vehicle is added to the cells (the “vehicle” or “no test compound-control”). The test compound may also be directly dissolved in medium that is added to the cells. In this case, only medium is added to the cells with regard to the “no-test-compound-control”. The cells are then incubated for 24 or 48 hours. Activity of SEAP is then measured using standard methods used in the field. For example, the medium containing shed SEAP-sorLA is transferred to a new test vessel and the substrate for SEAP is added. This will lead to a measurable signal, for example in form of emitted light or colour change, which will be measured (FIG. 5B).

### Results

The baseline reporter signal will be established by measuring the reporter signal in medium collected from cells, which were not exposed to the test compound (the “vehicle” or “no test compound-control”). In subsequent analysis this value, for example a mean value of several samples, may be regarded as a reporter signal of 100% (FIG. 5C). For each test compound, the reporter signal will be measured.

Test compounds which lead to an increase reporter signal are endosomal enhancers leading to enhanced sorLA-reporter endosomal processing and shedding (for example the sample indicated with grey arrows in FIG. 5C).

Test compounds which lead to a decreased reporter signal are endosomal repressors leading to decreased sorLA-reporter endosomal processing and shedding (for example the sample indicated with white arrows in FIG. 5C). The principle is summarized in FIG. 5D.

### Conclusions

Test compounds which elicit an increased reporter signal can be categorized as endosomal activators leading to increased endosomal processing and shedding of the sorLA-reporter-construct.

Test compounds which elicit a decreased reporter signal can be categorized as endosomal repressors leading to decreased endosomal processing and shedding of the sorLA-reporter-construct.

The above described assay using the sorLA-reporter construct as described in Example 1 and optionally positive and negative controls as in Example 2 provides an easy, straight forward and quantifiable approach to identify or screen for endosomal enhancers or endosomal repressors. The assay is suitable for high-throughput applications.

### Example 4: Linker Elongation

### Aim:

Optimization of the reporter activity using different linker sequences connecting the SEAP and SORLA partners.

### Materials and Methods

The linker between the SEAP and the SORLA VPS10p-domain will be extended from 2 amino acids (6 nucleotides) to 5 amino acids, by site-directed mutagenesis using a set of primers that carry a 9 nucleotides insert and the reporter construct as depicted in FIG. 1C (construct with 2 amino acids-linker) as template. This will result in the construct with an amino acid sequence of SEQ ID NO: 2 and FIG. 1C.

In a next experiment, a further elongation of the linker to 8 amino acids will performed by repeating the method and using a new set of primers again with 9 extra nucleotides insert (FIG. 1C). This will result in the construct with an amino acid sequence of SEQ ID NO: 3 and FIG. 1C.

In yet another experiment the entire 53 amino acid pro-peptide of SORLA (FIG. 1D). (corresponding to residues 29-81 of the SORLA sequence, Uniprot Q92673) will be included as linker, although the sequence RRKR⁸¹ that represents the cleavage site for Furin has been substituted with the GSGS sequence. This will result in the construct with an amino acid sequence of SEQ ID NO: 4 and FIG. 1D.

Any other length of the linker may be tested as well, for example any length between 1 and 53 amino acids. Criteria for selecting amino acid for the linkers may be that the amino acids are inert, water soluble and not sterically hampering, such as aromatic amino acids. For example, we will use and test linkers of sequences of SEQ ID NO: 12, SEQ ID NO: 13 and SEQ ID NO: 14. However, other lengths of linkers, and linkers of a different amino acid composition or sequence may be tested.

Activity of the set of SEAP reporter constructs will be determined by transient transfections of N2a cells and collection of 24 hour-conditioned medium, and will subsequently be quantified by the commercial QUANTI-Blue detection assay following suppliers guidelines.

### Results

By comparing the measured activity of the different SEAP reporter constructs, functional and/or optimal construct(s) will be identified.

### Conclusion

We will be able to identify the most optimal linker among the tested SEAP-SORLA reporter constructs.

### Example 5: Gaussia Luciferase Reporter

### Aim

As an alternative to use SEAP (~60 kDa; 550 amino acids) as fused reporter, we will test the smaller (~18 kDa; 180 amino acids) luciferase from Gaussia princeps, referred to as Gaussia Luciferase (Gluc), as fusion partner in SORLA reporter constructs (FIGS. 1 E, F ). Besides being the smallest luciferase, the Gluc is also ~2,000-fold more sensitive than standard luciferase reporters, and ~20,000-fold more sensitive than SEAP. To circumvent a potential problem of rapid decay of the luminescent signal from Gluc, we will use a mutated version of the Gluc with substitution of the oxidation-prone methionines M431 and M110 (eGluc-M431I;M110I) as described by Zadoo et al (Zadoo 2016).

Maguire et al also described as Gaussia Luciferase variant (GlucM43I) (Maguire 2009) which may be used. This variance with optimized properties may be referred to as enhanced Gluc (eGluc).

### Materials and Methods

A vector containing the enhanced Gaussia luciferase (eGluc) reporter attached with a linker (e.g. a linker of 2, 5 or 8 amino acids) to the MYOC gene (provided by John D. Hulleman) will serve as template where we will replace the MYOC insert with the SORL1 coding frame. This will result in the constructs with an amino acid sequence of SEQ ID NO: 6 and FIG. 1E; SEQ ID NO: 7 and FIG. 1E and SEQ ID NO: 8 and FIG. 1E. In a first PCR we will amplify the eGluc and vector - but without the MYOC part - using a set of primers that create sticky ends on both sites of the amplified fragment. In a second PCR we will amplify the SORLA region from the above described SORL1 expression plasmid with another set of primers that creates compatible sticky ends that match the sticky ends of the eGluc-vector construct. These two fragments will then be joined using the Gibson assembly method (a molecular cloning method allowing the ligation of multiple DNA fragments in a single, isothermal reaction). The SORLA-eGluc reporter construct with 2, 5, or 8 amino acid linker will be validated by standard DNA sequencing.

In another experiment, we will generate an eGluc-SORLA reporter construct where the linker will be made by the 53 amino acid SORLA pro-peptide with the sequence RRKR⁸¹ that represents the cleavage site for Furin has been substituted with the GSGS sequence. This will result in the constructs with an amino acid sequence of SEQ ID NO: 9 and FIG. 1F.

Activity of the eGluc-SORLA reporter constructs were tested by transient transfections of N2a cells, and quantified by the GLuc assay as described (Zadoo 2016), for example as exemplified in Example 3 and in FIG. 5.

### Results

By comparing the measured activity of the different Gluc reporter constructs, functional and/or optimal construct(s) will be identified.

### Conclusion

We will identify a Gluc-SORLA reporter construct that can be used for quantification of shed sSORLA in medium from transfected cells.

### Example 6: Pathogenic SORL1 Variants

### Aim

First, to demonstrate that introduction of mutations that correspond to known pathogenic missense variants in SORL1 decrease the amount of reporter activity in medium from transfected cells. Second, to test if a given SORL1 variant will respond to a drug that boosts endosomal activity.

### Materials and Methods

We will select a set of SORL1 missense variants that we have previously shown leads to decreased ssorLA levels shown by Western blotting of transfected N2a cells that shedding of mutated construct was compromised. Each mutation will be introduced into the reporter-construct of choice (i.e. optimized reporter construct, see Examples 1 to 5) by site-directed mutagenesis.

Cells expressing a given SORL1-variant reporter construct would be treated with a given drug and tested for activity by standard colorimetric assay.

### Results

Transfection of N2a cells with either a mutated or the wild-type reporter constructs will be performed using Fugene standard protocols, and medium will be harvested after 24 hours and subsequent used for determination of reporter activity as described above.

### Conclusion

We will find that a reporter construct carrying pathogenic SORL1 variants will have reduced reporter activity in the medium from transfected cells compared to the wild-type SORLA reporter construct. We will be able to determine if the mutant SORLA protein can respond to a drug that increase endosome activity.

Based on this we will be able to conclude that the reporter construct is suitable for screening pathogenicity of identified SORL1 variants. For example, a reporter construct carrying pathogenic SORL1 variants can serve as a negative control. This would ultimately serve the purpose to identify SORL1 carriers that can be enrolled in clinical trials for testing endosomal enhancers against AD.

### SEQUENCES

### Seq Id No: 1

### Human Sortilin-Related Receptor 1 (sorLA) Protein, Linker (2 Amino Acids) and a Reporter Fragment (SEAP)

as depicted in FIG. 1C

the linker is shown by underlining

MLLLL LLLGLRLQLS LGIIPVEEEN PDFWNREAAE ALGAAKKLQP
AQTAAKNLII FLGDGMGVST VTAARILKGQ KKDKLGPEIP LAMDRFPYVA
LSKTYNVDKH VPDSGATATA YLCGVKGNFQ TIGLSAAARF NQCNTTRGNE
VISVMNRAKK AGKSVGVVTT TRVQHASPAG TYAHTVNRNW YSDADVPASA
RQEGCQDIAT QLISNMDIDV ILGGGRKYMF RMGTPDPEYP DDYSQGGTRL
DGKNLVQEWL AKRQGARYVW NRTELMQASL DPSVTHLMGL FEPGDMKYEI
HRDSTLDPSL MEMTEAALRL LSRNPRGFFL FVEGGRIDHG HHESRAYRAL
TETIMFDDAI ERAGQLTSEE DTLSLVTADH SHVFSFGGYP LRGSSIFGLA
PGKARDRKAY TVLLYGNGPG YVLKDGARPD VTESESGSPE YRQQSAVPLD
EETHAGEDVA VFARGPQAHL VHGVQEQTFI AHVMAFAACL EPYTACDLAP
PAGTTDAAHP GLESAALQPEPI KVYGQVSLND
SHNQMVVHWA GEKSNVIVAL ARDSLALARP KSSDVYVSYD YGKSFKKISD
KLNFGLGNRS EAVIAQFYHS PADNKRYIFA DAYAQYLWIT FDFCNTLQGF
SIPFRAADLL LHSKASNLLL GFDRSHPNKQ LWKSDDFGQT WIMIQEHVKS
FSWGIDPYDK PNTIYIERHE PSGYSTVFRS TDFFQSRENQ EVILEEVRDF
QLRDKYMFAT KVVHLLGSEQ QSSVQLWVSF GRKPMRAAQF VTRHPINEYY
IADASEDQVF VCVSHSNNRT NLYISEAEGL KFSLSLENVL YYSPGGAGSD
TLVRYFANEP FADFHRVEGL QGVYIATLIN GSMNEENMRS VITFDKGGTW
EFLQAPAFTG YGEKINCELS QGCSLHLAQR LSQLLNLQLR RMPILSKESA
PGLIIATGSV GKNLASKTNV YISSSAGARW REALPGPHYY TWGDHGGIIT
AIAQGMETNE LKYSTNEGET WKTFIFSEKP VFVYGLLTEP GEKSTVFTIF
GSNKENVHSW LILQVNATDA LGVPCTENDY KLWSPSDERG NECLLGHKTV
FKRRTPHATC FNGEDFDRPV VVSNCSCTRE DYECDFGFKM SEDLSLEVCV
PDPEFSGKSY SPPVPCPVGS TYRRTRGYRK ISGDTCSGGD VEARLEGELV
PCPLAEENEF ILYAVRKSIY RYDLASGATE QLPLTGLRAA VALDFDYEHN
CLYWSDLALD VIQRLCLNGS TGQEVIINSG LETVEALAFE PLSQLLYWVD
AGFKKIEVAN PDGDFRLTIV NSSVLDRPRA LVLVPQEGVM FWTDWGDLKP
GIYRSNMDGS AAYHLVSEDV KWPNGISVDD QWIYWTDAYL ECIERITFSG
QQRSVILDNL PHPYAIAVFK NEIYWDDWSQ LSIFRASKYS GSQMEILANQ
LTGLMDMKIF YKGKNTGSNA CVPRPCSLLC LPKANNSRSC RCPEDVSSSV
LPSGDLMCDC PQGYQLKNNT CVKQENTCLR NQYRCSNGNC INSIWWCDFD
NDCGDMSDER NCPTTICDLD TQFRCQESGT CIPLSYKCDL EDDCGDNSDE
SHCEMHQCRS DEYNCSSGMC IRSSWVCDGD NDCRDWSDEA NCTAIYHTCE
ASNFQCRNGH CIPQRWACDG DTDCQDGSDE DPVNCEKKCN GFRCPNGTCI
PSSKHCDGLR DCSDGSDEQH CEPLCTHFMD FVCKNRQQCL FHSMVCDGII
QCRDGSDEDA AFAGCSQDPE FHKVCDEFGF QCQNGVCISL IWKCDGMDDC
GDYSDEANCE NPTEAPNCSR YFQFRCENGH CIPNRWKCDR ENDCGDWSDE
KDCGDSHILP FSTPGPSTCL PNYYRCSSGT CVMDTWVCDG YRDCADGSDE
EACPLLANVT AASTPTQLGR CDRFEFECHQ PKTCIPNWKR CDGHQDCQDG
RDEANCPTHS TLTCMSREFQ CEDGEACIVL SERCDGFLDC SDESDEKACS
DELTVYKVQN LQWTADFSGD VTLTWMRPKK MPSASCVYNV YYRVVGESIW
KTLETHSNKT NTVLKVLKPD TTYQVKVQVQ CLSKAHNTND FVTLRTPEGL
PDAPRNLQLS LPREAEGVIV GHWAPPIHTH GLIREYIVEY SRSGSKMWAS
QRAASNFTEI KNLLVNTLYT VRVAAVTSRG IGNWSDSKSI TTIKGKVIPP
PDIHIDSYGE NYLSFTLTME SDIKVNGYVV NLFWAFDTHK QERRTLNFRG
SILSHKVGNL TAHTSYEISA WAKTDLGDSP LAFEHVMTRG VRPPAPSLKA
KAINQTAVEC TWTGPRNVVY GIFYATSFLD LYRNPKSLTT SLHNKTVIVS
KDEQYLFLVR VVVPYQGPSS DYVVVKMIPD SRLPPRHLHV VHTGKTSVVI
KWESPYDSPD QDLLYAVAVK DLIRKTDRSY KVKSRNSTVE YTLNKLEPGG
KYHIIVQLGN MSKDSSIKIT TVSLSAPDAL KIITENDHVL LFWKSLALKE
KHFNESRGYE IHMFDSAMNI TAYLGNTTDN FFKISNLKMG HNYTFTVQAR
CLFGNQICGE PAILLYDELG SGADASATQA ARSTDVAAVV VPILFLILLS
LGVGFAILYT KHRRLQSSFT AFANSHYSSR LGSAIFSSGD DLGEDDEDAP
MITGFSDDVP MVIA

### Seq Id No: 2

### Human Sortilin-Related Receptor 1 (sorLA) Protein, Linker (5 Amino Acids) and a Reporter Fragment (SEAP)

as depicted in FIG. 1C

the linker is shown by underlining

MLLLL LLLGLRLQLS LGIIPVEEEN PDFWNREAAE ALGAAKKLQP
AQTAAKNLII FLGDGMGVST VTAARILKGQ KKDKLGPEIP LAMDRFPYVA
LSKTYNVDKH VPDSGATATA YLCGVKGNFQ TIGLSAAARF NQCNTTRGNE
VISVMNRAKK AGKSVGVVTT TRVQHASPAG TYAHTVNRNW YSDADVPASA
RQEGCQDIAT QLISNMDIDV ILGGGRKYMF RMGTPDPEYP DDYSQGGTRL
DGKNLVQEWL AKRQGARYVW NRTELMQASL DPSVTHLMGL FEPGDMKYEI
HRDSTLDPSL MEMTEAALRL LSRNPRGFFL FVEGGRIDHG HHESRAYRAL
TETIMFDDAI ERAGQLTSEE DTLSLVTADH SHVFSFGGYP LRGSSIFGLA
PGKARDRKAY TVLLYGNGPG YVLKDGARPD VTESESGSPE YRQQSAVPLD
EETHAGEDVA VFARGPQAHL VHGVQEQTFI AHVMAFAACL EPYTACDLAP
PAGTTDAAHP GLSRSESAALQPEPI KVYGQVSLND
SHNQMVVHWA GEKSNVIVAL ARDSLALARP KSSDVYVSYD YGKSFKKISD
KLNFGLGNRS EAVIAQFYHS PADNKRYIFA DAYAQYLWIT FDFCNTLQGF
SIPFRAADLL LHSKASNLLL GFDRSHPNKQ LWKSDDFGQT WIMIQEHVKS
FSWGIDPYDK PNTIYIERHE PSGYSTVFRS TDFFQSRENQ EVILEEVRDF
QLRDKYMFAT KVVHLLGSEQ QSSVQLWVSF GRKPMRAAQF VTRHPINEYY
IADASEDQVF VCVSHSNNRT NLYISEAEGL KFSLSLENVL YYSPGGAGSD
TLVRYFANEP FADFHRVEGL QGVYIATLIN GSMNEENMRS VITFDKGGTW
EFLQAPAFTG YGEKINCELS QGCSLHLAQR LSQLLNLQLR RMPILSKESA
PGLIIATGSV GKNLASKTNV YISSSAGARW REALPGPHYY TWGDHGGIIT
AIAQGMETNE LKYSTNEGET WKTFIFSEKP VFVYGLLTEP GEKSTVFTIF
GSNKENVHSW LILQVNATDA LGVPCTENDY KLWSPSDERG NECLLGHKTV
FKRRTPHATC FNGEDFDRPV VVSNCSCTRE DYECDFGFKM SEDLSLEVCV
PDPEFSGKSY SPPVPCPVGS TYRRTRGYRK ISGDTCSGGD VEARLEGELV
PCPLAEENEF ILYAVRKSIY RYDLASGATE QLPLTGLRAA VALDFDYEHN
CLYWSDLALD VIQRLCLNGS TGQEVIINSG LETVEALAFE PLSQLLYWVD
AGFKKIEVAN PDGDFRLTIV NSSVLDRPRA LVLVPQEGVM FWTDWGDLKP
GIYRSNMDGS AAYHLVSEDV KWPNGISVDD QWIYWTDAYL ECIERITFSG
QQRSVILDNL PHPYAIAVFK NEIYWDDWSQ LSIFRASKYS GSQMEILANQ
LTGLMDMKIF YKGKNTGSNA CVPRPCSLLC LPKANNSRSC RCPEDVSSSV
LPSGDLMCDC PQGYQLKNNT CVKQENTCLR NQYRCSNGNC INSIWWCDFD
NDCGDMSDER NCPTTICDLD TQFRCQESGT CIPLSYKCDL EDDCGDNSDE
SHCEMHQCRS DEYNCSSGMC IRSSWVCDGD NDCRDWSDEA NCTAIYHTCE
ASNFQCRNGH CIPQRWACDG DTDCQDGSDE DPVNCEKKCN GFRCPNGTCI
PSSKHCDGLR DCSDGSDEQH CEPLCTHFMD FVCKNRQQCL FHSMVCDGII
QCRDGSDEDA AFAGCSQDPE FHKVCDEFGF QCQNGVCISL IWKCDGMDDC
GDYSDEANCE NPTEAPNCSR YFQFRCENGH CIPNRWKCDR ENDCGDWSDE
KDCGDSHILP FSTPGPSTCL PNYYRCSSGT CVMDTWVCDG YRDCADGSDE
EACPLLANVT AASTPTQLGR CDRFEFECHQ PKTCIPNWKR CDGHQDCQDG
RDEANCPTHS TLTCMSREFQ CEDGEACIVL SERCDGFLDC SDESDEKACS
DELTVYKVQN LQWTADFSGD VTLTWMRPKK MPSASCVYNV YYRVVGESIW
KTLETHSNKT NTVLKVLKPD TTYQVKVQVQ CLSKAHNTND FVTLRTPEGL
PDAPRNLQLS LPREAEGVIV GHWAPPIHTH GLIREYIVEY SRSGSKMWAS
QRAASNFTEI KNLLVNTLYT VRVAAVTSRG IGNWSDSKSI TTIKGKVIPP
PDIHIDSYGE NYLSFTLTME SDIKVNGYVV NLFWAFDTHK QERRTLNFRG
SILSHKVGNL TAHTSYEISA WAKTDLGDSP LAFEHVMTRG VRPPAPSLKA
KAINQTAVEC TWTGPRNVVY GIFYATSFLD LYRNPKSLTT SLHNKTVIVS
KDEQYLFLVR VVVPYQGPSS DYVVVKMIPD SRLPPRHLHV VHTGKTSVVI
KWESPYDSPD QDLLYAVAVK DLIRKTDRSY KVKSRNSTVE YTLNKLEPGG
KYHIIVQLGN MSKDSSIKIT TVSLSAPDAL KIITENDHVL LFWKSLALKE
KHFNESRGYE IHMFDSAMNI TAYLGNTTDN FFKISNLKMG HNYTFTVQAR
CLFGNQICGE PAILLYDELG SGADASATQA ARSTDVAAVV VPILFLILLS
LGVGFAILYT KHRRLQSSFT AFANSHYSSR LGSAIFSSGD DLGEDDEDAP
MITGFSDDVP MVIA

### Seq Id No: 3

### Human Sortilin-Related Receptor 1 (sorLA) Protein, Linker (8 Amino Acids) and a Reporter Fragment (SEAP)

as depicted in FIG. 1C

the linker is shown by underlining

MLLLL LLLGLRLQLS LGIIPVEEEN PDFWNREAAE ALGAAKKLQP
AQTAAKNLII FLGDGMGVST VTAARILKGQ KKDKLGPEIP LAMDRFPYVA
LSKTYNVDKH VPDSGATATA YLCGVKGNFQ TIGLSAAARF NQCNTTRGNE
VISVMNRAKK AGKSVGVVTT TRVQHASPAG TYAHTVNRNW YSDADVPASA
RQEGCQDIAT QLISNMDIDV ILGGGRKYMF RMGTPDPEYP DDYSQGGTRL
DGKNLVQEWL AKRQGARYVW NRTELMQASL DPSVTHLMGL FEPGDMKYEI
HRDSTLDPSL MEMTEAALRL LSRNPRGFFL FVEGGRIDHG HHESRAYRAL
TETIMFDDAI ERAGQLTSEE DTLSLVTADH SHVFSFGGYP LRGSSIFGLA
PGKARDRKAY TVLLYGNGPG YVLKDGARPD VTESESGSPE YRQQSAVPLD
EETHAGEDVA VFARGPQAHL VHGVQEQTFI AHVMAFAACL EPYTACDLAP
PAGTTDAAHP GLSRSSGGESAALQPEPI KVYGQVSLND
SHNQMVVHWA GEKSNVIVAL ARDSLALARP KSSDVYVSYD YGKSFKKISD
KLNFGLGNRS EAVIAQFYHS PADNKRYIFA DAYAQYLWIT FDFCNTLQGF
SIPFRAADLL LHSKASNLLL GFDRSHPNKQ LWKSDDFGQT WIMIQEHVKS
FSWGIDPYDK PNTIYIERHE PSGYSTVFRS TDFFQSRENQ EVILEEVRDF
QLRDKYMFAT KVVHLLGSEQ QSSVQLWVSF GRKPMRAAQF VTRHPINEYY
IADASEDQVF VCVSHSNNRT NLYISEAEGL KFSLSLENVL YYSPGGAGSD
TLVRYFANEP FADFHRVEGL QGVYIATLIN GSMNEENMRS VITFDKGGTW
EFLQAPAFTG YGEKINCELS QGCSLHLAQR LSQLLNLQLR RMPILSKESA
PGLIIATGSV GKNLASKTNV YISSSAGARW REALPGPHYY TWGDHGGIIT
AIAQGMETNE LKYSTNEGET WKTFIFSEKP VFVYGLLTEP GEKSTVFTIF
GSNKENVHSW LILQVNATDA LGVPCTENDY KLWSPSDERG NECLLGHKTV
FKRRTPHATC FNGEDFDRPV VVSNCSCTRE DYECDFGFKM SEDLSLEVCV
PDPEFSGKSY SPPVPCPVGS TYRRTRGYRK ISGDTCSGGD VEARLEGELV
PCPLAEENEF ILYAVRKSIY RYDLASGATE QLPLTGLRAA VALDFDYEHN
CLYWSDLALD VIQRLCLNGS TGQEVIINSG LETVEALAFE PLSQLLYWVD
AGFKKIEVAN PDGDFRLTIV NSSVLDRPRA LVLVPQEGVM FWTDWGDLKP
GIYRSNMDGS AAYHLVSEDV KWPNGISVDD QWIYWTDAYL ECIERITFSG
QQRSVILDNL PHPYAIAVFK NEIYWDDWSQ LSIFRASKYS GSQMEILANQ
LTGLMDMKIF YKGKNTGSNA CVPRPCSLLC LPKANNSRSC RCPEDVSSSV
LPSGDLMCDC PQGYQLKNNT CVKQENTCLR NQYRCSNGNC INSIWWCDFD
NDCGDMSDER NCPTTICDLD TQFRCQESGT CIPLSYKCDL EDDCGDNSDE
SHCEMHQCRS DEYNCSSGMC IRSSWVCDGD NDCRDWSDEA NCTAIYHTCE
ASNFQCRNGH CIPQRWACDG DTDCQDGSDE DPVNCEKKCN GFRCPNGTCI
PSSKHCDGLR DCSDGSDEQH CEPLCTHFMD FVCKNRQQCL FHSMVCDGII
QCRDGSDEDA AFAGCSQDPE FHKVCDEFGF QCQNGVCISL IWKCDGMDDC
GDYSDEANCE NPTEAPNCSR YFQFRCENGH CIPNRWKCDR ENDCGDWSDE
KDCGDSHILP FSTPGPSTCL PNYYRCSSGT CVMDTWVCDG YRDCADGSDE
EACPLLANVT AASTPTQLGR CDRFEFECHQ PKTCIPNWKR CDGHQDCQDG
RDEANCPTHS TLTCMSREFQ CEDGEACIVL SERCDGFLDC SDESDEKACS
DELTVYKVQN LQWTADFSGD VTLTWMRPKK MPSASCVYNV YYRVVGESIW
KTLETHSNKT NTVLKVLKPD TTYQVKVQVQ CLSKAHNTND FVTLRTPEGL
PDAPRNLQLS LPREAEGVIV GHWAPPIHTH GLIREYIVEY SRSGSKMWAS
QRAASNFTEI KNLLVNTLYT VRVAAVTSRG IGNWSDSKSI TTIKGKVIPP
PDIHIDSYGE NYLSFTLTME SDIKVNGYVV NLFWAFDTHK QERRTLNFRG
SILSHKVGNL TAHTSYEISA WAKTDLGDSP LAFEHVMTRG VRPPAPSLKA
KAINQTAVEC TWTGPRNVVY GIFYATSFLD LYRNPKSLTT SLHNKTVIVS
KDEQYLFLVR VVVPYQGPSS DYVVVKMIPD SRLPPRHLHV VHTGKTSVVI
KWESPYDSPD QDLLYAVAVK DLIRKTDRSY KVKSRNSTVE YTLNKLEPGG
KYHIIVQLGN MSKDSSIKIT TVSLSAPDAL KIITENDHVL LFWKSLALKE
KHFNESRGYE IHMFDSAMNI TAYLGNTTDN FFKISNLKMG HNYTFTVQAR
CLFGNQICGE PAILLYDELG SGADASATQA ARSTDVAAVV VPILFLILLS
LGVGFAILYT KHRRLQSSFT AFANSHYSSR LGSAIFSSGD DLGEDDEDAP
MITGFSDDVP MVIA

### Seq Id No: 4

### Human Sortilin-Related Receptor 1 (sorLA) Protein, Linker (53 Amino Acids) and a Reporter Fragment (SEAP)

as depicted in FIG. 1D

the linker is shown by underlining

MLLLL LLLGLRLQLS LGIIPVEEEN PDFWNREAAE ALGAAKKLQP
AQTAAKNLII FLGDGMGVST VTAARILKGQ KKDKLGPEIP LAMDRFPYVA
LSKTYNVDKH VPDSGATATA YLCGVKGNFQ TIGLSAAARF NQCNTTRGNE
VISVMNRAKK AGKSVGVVTT TRVQHASPAG TYAHTVNRNW YSDADVPASA
RQEGCQDIAT QLISNMDIDV ILGGGRKYMF RMGTPDPEYP DDYSQGGTRL
DGKNLVQEWL AKRQGARYVW NRTELMQASL DPSVTHLMGL FEPGDMKYEI
HRDSTLDPSL MEMTEAALRL LSRNPRGFFL FVEGGRIDHG HHESRAYRAL
TETIMFDDAI ERAGQLTSEE DTLSLVTADH SHVFSFGGYP LRGSSIFGLA
PGKARDRKAY TVLLYGNGPG YVLKDGARPD VTESESGSPE YRQQSAVPLD
EETHAGEDVA VFARGPQAHL VHGVQEQTFI AHVMAFAACL EPYTACDLAP
PAGTTDAAHP G
EVWTQRLHGGSAPLPQDRGFLVVQGDPRELRLWARGDARGASRADEKPLGSGSSAALQPEPI
KVYGQVSLND
SHNQMVVHWA GEKSNVIVAL ARDSLALARP KSSDVYVSYD YGKSFKKISD
KLNFGLGNRS EAVIAQFYHS PADNKRYIFA DAYAQYLWIT FDFCNTLQGF
SIPFRAADLL LHSKASNLLL GFDRSHPNKQ LWKSDDFGQT WIMIQEHVKS
FSWGIDPYDK PNTIYIERHE PSGYSTVFRS TDFFQSRENQ EVILEEVRDF
QLRDKYMFAT KVVHLLGSEQ QSSVQLWVSF GRKPMRAAQF VTRHPINEYY
IADASEDQVF VCVSHSNNRT NLYISEAEGL KFSLSLENVL YYSPGGAGSD
TLVRYFANEP FADFHRVEGL QGVYIATLIN GSMNEENMRS VITFDKGGTW
EFLQAPAFTG YGEKINCELS QGCSLHLAQR LSQLLNLQLR RMPILSKESA
PGLIIATGSV GKNLASKTNV YISSSAGARW REALPGPHYY TWGDHGGIIT
AIAQGMETNE LKYSTNEGET WKTFIFSEKP VFVYGLLTEP GEKSTVFTIF
GSNKENVHSW LILQVNATDA LGVPCTENDY KLWSPSDERG NECLLGHKTV
FKRRTPHATC FNGEDFDRPV VVSNCSCTRE DYECDFGFKM SEDLSLEVCV
PDPEFSGKSY SPPVPCPVGS TYRRTRGYRK ISGDTCSGGD VEARLEGELV
PCPLAEENEF ILYAVRKSIY RYDLASGATE QLPLTGLRAA VALDFDYEHN
CLYWSDLALD VIQRLCLNGS TGQEVIINSG LETVEALAFE PLSQLLYWVD
AGFKKIEVAN PDGDFRLTIV NSSVLDRPRA LVLVPQEGVM FWTDWGDLKP
GIYRSNMDGS AAYHLVSEDV KWPNGISVDD QWIYWTDAYL ECIERITFSG
QQRSVILDNL PHPYAIAVFK NEIYWDDWSQ LSIFRASKYS GSQMEILANQ
LTGLMDMKIF YKGKNTGSNA CVPRPCSLLC LPKANNSRSC RCPEDVSSSV
LPSGDLMCDC PQGYQLKNNT CVKQENTCLR NQYRCSNGNC INSIWWCDFD
NDCGDMSDER NCPTTICDLD TQFRCQESGT CIPLSYKCDL EDDCGDNSDE
SHCEMHQCRS DEYNCSSGMC IRSSWVCDGD NDCRDWSDEA NCTAIYHTCE
ASNFQCRNGH CIPQRWACDG DTDCQDGSDE DPVNCEKKCN GFRCPNGTCI
PSSKHCDGLR DCSDGSDEQH CEPLCTHFMD FVCKNRQQCL FHSMVCDGII
QCRDGSDEDA AFAGCSQDPE FHKVCDEFGF QCQNGVCISL IWKCDGMDDC
GDYSDEANCE NPTEAPNCSR YFQFRCENGH CIPNRWKCDR ENDCGDWSDE
KDCGDSHILP FSTPGPSTCL PNYYRCSSGT CVMDTWVCDG YRDCADGSDE
EACPLLANVT AASTPTQLGR CDRFEFECHQ PKTCIPNWKR CDGHQDCQDG
RDEANCPTHS TLTCMSREFQ CEDGEACIVL SERCDGFLDC SDESDEKACS
DELTVYKVQN LQWTADFSGD VTLTWMRPKK MPSASCVYNV YYRVVGESIW
KTLETHSNKT NTVLKVLKPD TTYQVKVQVQ CLSKAHNTND FVTLRTPEGL
PDAPRNLQLS LPREAEGVIV GHWAPPIHTH GLIREYIVEY SRSGSKMWAS
QRAASNFTEI KNLLVNTLYT VRVAAVTSRG IGNWSDSKSI TTIKGKVIPP
PDIHIDSYGE NYLSFTLTME SDIKVNGYVV NLFWAFDTHK QERRTLNFRG
SILSHKVGNL TAHTSYEISA WAKTDLGDSP LAFEHVMTRG VRPPAPSLKA
KAINQTAVEC TWTGPRNVVY GIFYATSFLD LYRNPKSLTT SLHNKTVIVS
KDEQYLFLVR VVVPYQGPSS DYVVVKMIPD SRLPPRHLHV VHTGKTSVVI
KWESPYDSPD QDLLYAVAVK DLIRKTDRSY KVKSRNSTVE YTLNKLEPGG
KYHIIVQLGN MSKDSSIKIT TVSLSAPDAL KIITENDHVL LFWKSLALKE
KHFNESRGYE IHMFDSAMNI TAYLGNTTDN FFKISNLKMG HNYTFTVQAR
CLFGNQICGE PAILLYDELG SGADASATQA ARSTDVAAVV VPILFLILLS
LGVGFAILYT KHRRLQSSFT AFANSHYSSR LGSAIFSSGD DLGEDDEDAP
MITGFSDDVP MVIA

### Seq Id No: 5

### Human Sortilin-Related Receptor 1 (sorLA) Protein and a Reporter Fragment (SEAP) (No Linker)

as depicted in FIG. 1B

MLLLL LLLGLRLQLS LGIIPVEEEN PDFWNREAAE ALGAAKKLQP
AQTAAKNLII FLGDGMGVST VTAARILKGQ KKDKLGPEIP LAMDRFPYVA
LSKTYNVDKH VPDSGATATA YLCGVKGNFQ TIGLSAAARF NQCNTTRGNE
VISVMNRAKK AGKSVGVVTT TRVQHASPAG TYAHTVNRNW YSDADVPASA
RQEGCQDIAT QLISNMDIDV ILGGGRKYMF RMGTPDPEYP DDYSQGGTRL
DGKNLVQEWL AKRQGARYVW NRTELMQASL DPSVTHLMGL FEPGDMKYEI
HRDSTLDPSL MEMTEAALRL LSRNPRGFFL FVEGGRIDHG HHESRAYRAL
TETIMFDDAI ERAGQLTSEE DTLSLVTADH SHVFSFGGYP LRGSSIFGLA
PGKARDRKAY TVLLYGNGPG YVLKDGARPD VTESESGSPE YRQQSAVPLD
EETHAGEDVA VFARGPQAHL VHGVQEQTFI AHVMAFAACL EPYTACDLAP
PAGTTDAAHP GSAALQPEPI KVYGQVSLND
SHNQMVVHWA GEKSNVIVAL ARDSLALARP KSSDVYVSYD YGKSFKKISD
KLNFGLGNRS EAVIAQFYHS PADNKRYIFA DAYAQYLWIT FDFCNTLQGF
SIPFRAADLL LHSKASNLLL GFDRSHPNKQ LWKSDDFGQT WIMIQEHVKS
FSWGIDPYDK PNTIYIERHE PSGYSTVFRS TDFFQSRENQ EVILEEVRDF
QLRDKYMFAT KVVHLLGSEQ QSSVQLWVSF GRKPMRAAQF VTRHPINEYY
IADASEDQVF VCVSHSNNRT NLYISEAEGL KFSLSLENVL YYSPGGAGSD
TLVRYFANEP FADFHRVEGL QGVYIATLIN GSMNEENMRS VITFDKGGTW
EFLQAPAFTG YGEKINCELS QGCSLHLAQR LSQLLNLQLR RMPILSKESA
PGLIIATGSV GKNLASKTNV YISSSAGARW REALPGPHYY TWGDHGGIIT
AIAQGMETNE LKYSTNEGET WKTFIFSEKP VFVYGLLTEP GEKSTVFTIF
GSNKENVHSW LILQVNATDA LGVPCTENDY KLWSPSDERG NECLLGHKTV
FKRRTPHATC FNGEDFDRPV VVSNCSCTRE DYECDFGFKM SEDLSLEVCV
PDPEFSGKSY SPPVPCPVGS TYRRTRGYRK ISGDTCSGGD VEARLEGELV
PCPLAEENEF ILYAVRKSIY RYDLASGATE QLPLTGLRAA VALDFDYEHN
CLYWSDLALD VIQRLCLNGS TGQEVIINSG LETVEALAFE PLSQLLYWVD
AGFKKIEVAN PDGDFRLTIV NSSVLDRPRA LVLVPQEGVM FWTDWGDLKP
GIYRSNMDGS AAYHLVSEDV KWPNGISVDD QWIYWTDAYL ECIERITFSG
QQRSVILDNL PHPYAIAVFK NEIYWDDWSQ LSIFRASKYS GSQMEILANQ
LTGLMDMKIF YKGKNTGSNA CVPRPCSLLC LPKANNSRSC RCPEDVSSSV
LPSGDLMCDC PQGYQLKNNT CVKQENTCLR NQYRCSNGNC INSIWWCDFD
NDCGDMSDER NCPTTICDLD TQFRCQESGT CIPLSYKCDL EDDCGDNSDE
SHCEMHQCRS DEYNCSSGMC IRSSWVCDGD NDCRDWSDEA NCTAIYHTCE
ASNFQCRNGH CIPQRWACDG DTDCQDGSDE DPVNCEKKCN GFRCPNGTCI
PSSKHCDGLR DCSDGSDEQH CEPLCTHFMD FVCKNRQQCL FHSMVCDGII
QCRDGSDEDA AFAGCSQDPE FHKVCDEFGF QCQNGVCISL IWKCDGMDDC
GDYSDEANCE NPTEAPNCSR YFQFRCENGH CIPNRWKCDR ENDCGDWSDE
KDCGDSHILP FSTPGPSTCL PNYYRCSSGT CVMDTWVCDG YRDCADGSDE
EACPLLANVT AASTPTQLGR CDRFEFECHQ PKTCIPNWKR CDGHQDCQDG
RDEANCPTHS TLTCMSREFQ CEDGEACIVL SERCDGFLDC SDESDEKACS
DELTVYKVQN LQWTADFSGD VTLTWMRPKK MPSASCVYNV YYRVVGESIW
KTLETHSNKT NTVLKVLKPD TTYQVKVQVQ CLSKAHNTND FVTLRTPEGL
PDAPRNLQLS LPREAEGVIV GHWAPPIHTH GLIREYIVEY SRSGSKMWAS
QRAASNFTEI KNLLVNTLYT VRVAAVTSRG IGNWSDSKSI TTIKGKVIPP
PDIHIDSYGE NYLSFTLTME SDIKVNGYVV NLFWAFDTHK QERRTLNFRG
SILSHKVGNL TAHTSYEISA WAKTDLGDSP LAFEHVMTRG VRPPAPSLKA
KAINQTAVEC TWTGPRNVVY GIFYATSFLD LYRNPKSLTT SLHNKTVIVS
KDEQYLFLVR VVVPYQGPSS DYVVVKMIPD SRLPPRHLHV VHTGKTSVVI
KWESPYDSPD QDLLYAVAVK DLIRKTDRSY KVKSRNSTVE YTLNKLEPGG
KYHIIVQLGN MSKDSSIKIT TVSLSAPDAL KIITENDHVL LFWKSLALKE
KHFNESRGYE IHMFDSAMNI TAYLGNTTDN FFKISNLKMG HNYTFTVQAR
CLFGNQICGE PAILLYDELG SGADASATQA ARSTDVAAVV VPILFLILLS
LGVGFAILYT KHRRLQSSFT AFANSHYSSR LGSAIFSSGD DLGEDDEDAP
MITGFSDDVP MVIA

### Seq Id No: 6

### Human Sortilin-Related Receptor 1 (sorLA) Protein, Linker (2 Amino Acids) and a Reporter Fragment (eGluc)

as depicted in FIG. 1E

the linker is shown by underlining

KPTENNEDFNIVAVASNFATTDLDADRGKLPGKKLPLEVLKEIEANARKAGCTRGCLICLSHIK
CTPKMKKFIPGRCHTYEGDKESAQGGIGEAIVDIPEIPGFKDLEPIEQFIAQVDLCVDCTTGCL
KGLANVQCSDLLKKWLPQRCATFASKIQGQVDKIKGAGGDLESAALQPEPI KVYGQVSLND
SHNQMVVHWA GEKSNVIVAL ARDSLALARP KSSDVYVSYD YGKSFKKISD
KLNFGLGNRS EAVIAQFYHS PADNKRYIFA DAYAQYLWIT FDFCNTLQGF
SIPFRAADLL LHSKASNLLL GFDRSHPNKQ LWKSDDFGQT WIMIQEHVKS
FSWGIDPYDK PNTIYIERHE PSGYSTVFRS TDFFQSRENQ EVILEEVRDF
QLRDKYMFAT KVVHLLGSEQ QSSVQLWVSF GRKPMRAAQF VTRHPINEYY
IADASEDQVF VCVSHSNNRT NLYISEAEGL KFSLSLENVL YYSPGGAGSD
TLVRYFANEP FADFHRVEGL QGVYIATLIN GSMNEENMRS VITFDKGGTW
EFLQAPAFTG YGEKINCELS QGCSLHLAQR LSQLLNLQLR RMPILSKESA
PGLIIATGSV GKNLASKTNV YISSSAGARW REALPGPHYY TWGDHGGIIT
AIAQGMETNE LKYSTNEGET WKTFIFSEKP VFVYGLLTEP GEKSTVFTIF
GSNKENVHSW LILQVNATDA LGVPCTENDY KLWSPSDERG NECLLGHKTV
FKRRTPHATC FNGEDFDRPV VVSNCSCTRE DYECDFGFKM SEDLSLEVCV
PDPEFSGKSY SPPVPCPVGS TYRRTRGYRK ISGDTCSGGD VEARLEGELV
PCPLAEENEF ILYAVRKSIY RYDLASGATE QLPLTGLRAA VALDFDYEHN
CLYWSDLALD VIQRLCLNGS TGQEVIINSG LETVEALAFE PLSQLLYWVD
AGFKKIEVAN PDGDFRLTIV NSSVLDRPRA LVLVPQEGVM FWTDWGDLKP
GIYRSNMDGS AAYHLVSEDV KWPNGISVDD QWIYWTDAYL ECIERITFSG
QQRSVILDNL PHPYAIAVFK NEIYWDDWSQ LSIFRASKYS GSQMEILANQ
LTGLMDMKIF YKGKNTGSNA CVPRPCSLLC LPKANNSRSC RCPEDVSSSV
LPSGDLMCDC PQGYQLKNNT CVKQENTCLR NQYRCSNGNC INSIWWCDFD
NDCGDMSDER NCPTTICDLD TQFRCQESGT CIPLSYKCDL EDDCGDNSDE
SHCEMHQCRS DEYNCSSGMC IRSSWVCDGD NDCRDWSDEA NCTAIYHTCE
ASNFQCRNGH CIPQRWACDG DTDCQDGSDE DPVNCEKKCN GFRCPNGTCI
PSSKHCDGLR DCSDGSDEQH CEPLCTHFMD FVCKNRQQCL FHSMVCDGII
QCRDGSDEDA AFAGCSQDPE FHKVCDEFGF QCQNGVCISL IWKCDGMDDC
GDYSDEANCE NPTEAPNCSR YFQFRCENGH CIPNRWKCDR ENDCGDWSDE
KDCGDSHILP FSTPGPSTCL PNYYRCSSGT CVMDTWVCDG YRDCADGSDE
EACPLLANVT AASTPTQLGR CDRFEFECHQ PKTCIPNWKR CDGHQDCQDG
RDEANCPTHS TLTCMSREFQ CEDGEACIVL SERCDGFLDC SDESDEKACS
DELTVYKVQN LQWTADFSGD VTLTWMRPKK MPSASCVYNV YYRVVGESIW
KTLETHSNKT NTVLKVLKPD TTYQVKVQVQ CLSKAHNTND FVTLRTPEGL
PDAPRNLQLS LPREAEGVIV GHWAPPIHTH GLIREYIVEY SRSGSKMWAS
QRAASNFTEI KNLLVNTLYT VRVAAVTSRG IGNWSDSKSI TTIKGKVIPP
PDIHIDSYGE NYLSFTLTME SDIKVNGYVV NLFWAFDTHK QERRTLNFRG
SILSHKVGNL TAHTSYEISA WAKTDLGDSP LAFEHVMTRG VRPPAPSLKA
KAINQTAVEC TWTGPRNVVY GIFYATSFLD LYRNPKSLTT SLHNKTVIVS
KDEQYLFLVR VVVPYQGPSS DYVVVKMIPD SRLPPRHLHV VHTGKTSVVI
KWESPYDSPD QDLLYAVAVK DLIRKTDRSY KVKSRNSTVE YTLNKLEPGG
KYHIIVQLGN MSKDSSIKIT TVSLSAPDAL KIITENDHVL LFWKSLALKE
KHFNESRGYE IHMFDSAMNI TAYLGNTTDN FFKISNLKMG HNYTFTVQAR
CLFGNQICGE PAILLYDELG SGADASATQA ARSTDVAAVV VPILFLILLS
LGVGFAILYT KHRRLQSSFT AFANSHYSSR LGSAIFSSGD DLGEDDEDAP
MITGFSDDVP MVIA

### Seq Id No: 7

### Human Sortilin-Related Receptor 1 (sorLA) Protein, Linker (5 Amino Acids) and a Reporter Fragment (eGluc)

as depicted in FIG. 1E

the linker is shown by underlining

KPTENNEDFNIVAVASNFATTDLDADRGKLPGKKLPLEVLKEIEANARKAGCTRGCLICLSHIK
CTPKMKKFIPGRCHTYEGDKESAQGGIGEAIVDIPEIPGFKDLEPIEQFIAQVDLCVDCTTGCL
KGLANVQCSDLLKKWLPQRCATFASKIQGQVDKIKGAGGDLSRSESAALQPEPI
KVYGQVSLND
SHNQMVVHWA GEKSNVIVAL ARDSLALARP KSSDVYVSYD YGKSFKKISD
KLNFGLGNRS EAVIAQFYHS PADNKRYIFA DAYAQYLWIT FDFCNTLQGF
SIPFRAADLL LHSKASNLLL GFDRSHPNKQ LWKSDDFGQT WIMIQEHVKS
FSWGIDPYDK PNTIYIERHE PSGYSTVFRS TDFFQSRENQ EVILEEVRDF
QLRDKYMFAT KVVHLLGSEQ QSSVQLWVSF GRKPMRAAQF VTRHPINEYY
IADASEDQVF VCVSHSNNRT NLYISEAEGL KFSLSLENVL YYSPGGAGSD
TLVRYFANEP FADFHRVEGL QGVYIATLIN GSMNEENMRS VITFDKGGTW
EFLQAPAFTG YGEKINCELS QGCSLHLAQR LSQLLNLQLR RMPILSKESA
PGLIIATGSV GKNLASKTNV YISSSAGARW REALPGPHYY TWGDHGGIIT
AIAQGMETNE LKYSTNEGET WKTFIFSEKP VFVYGLLTEP GEKSTVFTIF
GSNKENVHSW LILQVNATDA LGVPCTENDY KLWSPSDERG NECLLGHKTV
FKRRTPHATC FNGEDFDRPV VVSNCSCTRE DYECDFGFKM SEDLSLEVCV
PDPEFSGKSY SPPVPCPVGS TYRRTRGYRK ISGDTCSGGD VEARLEGELV
PCPLAEENEF ILYAVRKSIY RYDLASGATE QLPLTGLRAA VALDFDYEHN
CLYWSDLALD VIQRLCLNGS TGQEVIINSG LETVEALAFE PLSQLLYWVD
AGFKKIEVAN PDGDFRLTIV NSSVLDRPRA LVLVPQEGVM FWTDWGDLKP
GIYRSNMDGS AAYHLVSEDV KWPNGISVDD QWIYWTDAYL ECIERITFSG
QQRSVILDNL PHPYAIAVFK NEIYWDDWSQ LSIFRASKYS GSQMEILANQ
LTGLMDMKIF YKGKNTGSNA CVPRPCSLLC LPKANNSRSC RCPEDVSSSV
LPSGDLMCDC PQGYQLKNNT CVKQENTCLR NQYRCSNGNC INSIWWCDFD
NDCGDMSDER NCPTTICDLD TQFRCQESGT CIPLSYKCDL EDDCGDNSDE
SHCEMHQCRS DEYNCSSGMC IRSSWVCDGD NDCRDWSDEA NCTAIYHTCE
ASNFQCRNGH CIPQRWACDG DTDCQDGSDE DPVNCEKKCN GFRCPNGTCI
PSSKHCDGLR DCSDGSDEQH CEPLCTHFMD FVCKNRQQCL FHSMVCDGII
QCRDGSDEDA AFAGCSQDPE FHKVCDEFGF QCQNGVCISL IWKCDGMDDC
GDYSDEANCE NPTEAPNCSR YFQFRCENGH CIPNRWKCDR ENDCGDWSDE
KDCGDSHILP FSTPGPSTCL PNYYRCSSGT CVMDTWVCDG YRDCADGSDE
EACPLLANVT AASTPTQLGR CDRFEFECHQ PKTCIPNWKR CDGHQDCQDG
RDEANCPTHS TLTCMSREFQ CEDGEACIVL SERCDGFLDC SDESDEKACS
DELTVYKVQN LQWTADFSGD VTLTWMRPKK MPSASCVYNV YYRVVGESIW
KTLETHSNKT NTVLKVLKPD TTYQVKVQVQ CLSKAHNTND FVTLRTPEGL
PDAPRNLQLS LPREAEGVIV GHWAPPIHTH GLIREYIVEY SRSGSKMWAS
QRAASNFTEI KNLLVNTLYT VRVAAVTSRG IGNWSDSKSI TTIKGKVIPP
PDIHIDSYGE NYLSFTLTME SDIKVNGYVV NLFWAFDTHK QERRTLNFRG
SILSHKVGNL TAHTSYEISA WAKTDLGDSP LAFEHVMTRG VRPPAPSLKA
KAINQTAVEC TWTGPRNVVY GIFYATSFLD LYRNPKSLTT SLHNKTVIVS
KDEQYLFLVR VVVPYQGPSS DYVVVKMIPD SRLPPRHLHV VHTGKTSVVI
KWESPYDSPD QDLLYAVAVK DLIRKTDRSY KVKSRNSTVE YTLNKLEPGG
KYHIIVQLGN MSKDSSIKIT TVSLSAPDAL KIITENDHVL LFWKSLALKE
KHFNESRGYE IHMFDSAMNI TAYLGNTTDN FFKISNLKMG HNYTFTVQAR
CLFGNQICGE PAILLYDELG SGADASATQA ARSTDVAAVV VPILFLILLS
LGVGFAILYT KHRRLQSSFT AFANSHYSSR LGSAIFSSGD DLGEDDEDAP
MITGFSDDVP MVIA

### Seq Id No: 8

### Human Sortilin-Related Receptor 1 (sorLA) Protein, Linker (8 Amino Acids) and a Reporter Fragment (eGluc)

as depicted in FIG. 1E

the linker is shown by underlining

KPTENNEDFNIVAVASNFATTDLDADRGKLPGKKLPLEVLKEIEANARKAGCTRGCLICLSHIK
CTPKMKKFIPGRCHTYEGDKESAQGGIGEAIVDIPEIPGFKDLEPIEQFIAQVDLCVDCTTGCL
KGLANVQCSDLLKKWLPQRCATFASKIQGQVDKIKGAGGDLSRSSGGESAALQPEPI
KVYGQVSLND
SHNQMVVHWA GEKSNVIVAL ARDSLALARP KSSDVYVSYD YGKSFKKISD
KLNFGLGNRS EAVIAQFYHS PADNKRYIFA DAYAQYLWIT FDFCNTLQGF
SIPFRAADLL LHSKASNLLL GFDRSHPNKQ LWKSDDFGQT WIMIQEHVKS
FSWGIDPYDK PNTIYIERHE PSGYSTVFRS TDFFQSRENQ EVILEEVRDF
QLRDKYMFAT KVVHLLGSEQ QSSVQLWVSF GRKPMRAAQF VTRHPINEYY
IADASEDQVF VCVSHSNNRT NLYISEAEGL KFSLSLENVL YYSPGGAGSD
TLVRYFANEP FADFHRVEGL QGVYIATLIN GSMNEENMRS VITFDKGGTW
EFLQAPAFTG YGEKINCELS QGCSLHLAQR LSQLLNLQLR RMPILSKESA
PGLIIATGSV GKNLASKTNV YISSSAGARW REALPGPHYY TWGDHGGIIT
AIAQGMETNE LKYSTNEGET WKTFIFSEKP VFVYGLLTEP GEKSTVFTIF
GSNKENVHSW LILQVNATDA LGVPCTENDY KLWSPSDERG NECLLGHKTV
FKRRTPHATC FNGEDFDRPV VVSNCSCTRE DYECDFGFKM SEDLSLEVCV
PDPEFSGKSY SPPVPCPVGS TYRRTRGYRK ISGDTCSGGD VEARLEGELV
PCPLAEENEF ILYAVRKSIY RYDLASGATE QLPLTGLRAA VALDFDYEHN
CLYWSDLALD VIQRLCLNGS TGQEVIINSG LETVEALAFE PLSQLLYWVD
AGFKKIEVAN PDGDFRLTIV NSSVLDRPRA LVLVPQEGVM FWTDWGDLKP
GIYRSNMDGS AAYHLVSEDV KWPNGISVDD QWIYWTDAYL ECIERITFSG
QQRSVILDNL PHPYAIAVFK NEIYWDDWSQ LSIFRASKYS GSQMEILANQ
LTGLMDMKIF YKGKNTGSNA CVPRPCSLLC LPKANNSRSC RCPEDVSSSV
LPSGDLMCDC PQGYQLKNNT CVKQENTCLR NQYRCSNGNC INSIWWCDFD
NDCGDMSDER NCPTTICDLD TQFRCQESGT CIPLSYKCDL EDDCGDNSDE
SHCEMHQCRS DEYNCSSGMC IRSSWVCDGD NDCRDWSDEA NCTAIYHTCE
ASNFQCRNGH CIPQRWACDG DTDCQDGSDE DPVNCEKKCN GFRCPNGTCI
PSSKHCDGLR DCSDGSDEQH CEPLCTHFMD FVCKNRQQCL FHSMVCDGII
QCRDGSDEDA AFAGCSQDPE FHKVCDEFGF QCQNGVCISL IWKCDGMDDC
GDYSDEANCE NPTEAPNCSR YFQFRCENGH CIPNRWKCDR ENDCGDWSDE
KDCGDSHILP FSTPGPSTCL PNYYRCSSGT CVMDTWVCDG YRDCADGSDE
EACPLLANVT AASTPTQLGR CDRFEFECHQ PKTCIPNWKR CDGHQDCQDG
RDEANCPTHS TLTCMSREFQ CEDGEACIVL SERCDGFLDC SDESDEKACS
DELTVYKVQN LQWTADFSGD VTLTWMRPKK MPSASCVYNV YYRVVGESIW
KTLETHSNKT NTVLKVLKPD TTYQVKVQVQ CLSKAHNTND FVTLRTPEGL
PDAPRNLQLS LPREAEGVIV GHWAPPIHTH GLIREYIVEY SRSGSKMWAS
QRAASNFTEI KNLLVNTLYT VRVAAVTSRG IGNWSDSKSI TTIKGKVIPP
PDIHIDSYGE NYLSFTLTME SDIKVNGYVV NLFWAFDTHK QERRTLNFRG
SILSHKVGNL TAHTSYEISA WAKTDLGDSP LAFEHVMTRG VRPPAPSLKA
KAINQTAVEC TWTGPRNVVY GIFYATSFLD LYRNPKSLTT SLHNKTVIVS
KDEQYLFLVR VVVPYQGPSS DYVVVKMIPD SRLPPRHLHV VHTGKTSVVI
KWESPYDSPD QDLLYAVAVK DLIRKTDRSY KVKSRNSTVE YTLNKLEPGG
KYHIIVQLGN MSKDSSIKIT TVSLSAPDAL KIITENDHVL LFWKSLALKE
KHFNESRGYE IHMFDSAMNI TAYLGNTTDN FFKISNLKMG HNYTFTVQAR
CLFGNQICGE PAILLYDELG SGADASATQA ARSTDVAAVV VPILFLILLS
LGVGFAILYT KHRRLQSSFT AFANSHYSSR LGSAIFSSGD DLGEDDEDAP
MITGFSDDVP MVIA

### Seq Id No: 9

### Human Sortilin-Related Receptor 1 (sorLA) Protein, Linker (53 Amino Acids) and a Reporter Fragment (eGluc)

as depicted in FIG. 1F

KPTENNEDFNIVAVASNFATTDLDADRGKLPGKKLPLEVLKEIEANARKAGCTRGCLICLSHIK
CTPKMKKFIPGRCHTYEGDKESAQGGIGEAIVDIPEIPGFKDLEPIEQFIAQVDLCVDCTTGCL
KGLANVQCSDLLKKWLPQRCATFASKIQGQVDKIKGAGGD
EVWTQRLHGGSAPLPQDRGFLVVQGDPRELRLWARGDARGASRADEKPLGSGS
SAALQPEPI KVYGQVSLND
SHNQMVVHWA GEKSNVIVAL ARDSLALARP KSSDVYVSYD YGKSFKKISD
KLNFGLGNRS EAVIAQFYHS PADNKRYIFA DAYAQYLWIT FDFCNTLQGF
SIPFRAADLL LHSKASNLLL GFDRSHPNKQ LWKSDDFGQT WIMIQEHVKS
FSWGIDPYDK PNTIYIERHE PSGYSTVFRS TDFFQSRENQ EVILEEVRDF
QLRDKYMFAT KVVHLLGSEQ QSSVQLWVSF GRKPMRAAQF VTRHPINEYY
IADASEDQVF VCVSHSNNRT NLYISEAEGL KFSLSLENVL YYSPGGAGSD
TLVRYFANEP FADFHRVEGL QGVYIATLIN GSMNEENMRS VITFDKGGTW
EFLQAPAFTG YGEKINCELS QGCSLHLAQR LSQLLNLQLR RMPILSKESA
PGLIIATGSV GKNLASKTNV YISSSAGARW REALPGPHYY TWGDHGGIIT
AIAQGMETNE LKYSTNEGET WKTFIFSEKP VFVYGLLTEP GEKSTVFTIF
GSNKENVHSW LILQVNATDA LGVPCTENDY KLWSPSDERG NECLLGHKTV
FKRRTPHATC FNGEDFDRPV VVSNCSCTRE DYECDFGFKM SEDLSLEVCV
PDPEFSGKSY SPPVPCPVGS TYRRTRGYRK ISGDTCSGGD VEARLEGELV
PCPLAEENEF ILYAVRKSIY RYDLASGATE QLPLTGLRAA VALDFDYEHN
CLYWSDLALD VIQRLCLNGS TGQEVIINSG LETVEALAFE PLSQLLYWVD
AGFKKIEVAN PDGDFRLTIV NSSVLDRPRA LVLVPQEGVM FWTDWGDLKP
GIYRSNMDGS AAYHLVSEDV KWPNGISVDD QWIYWTDAYL ECIERITFSG
QQRSVILDNL PHPYAIAVFK NEIYWDDWSQ LSIFRASKYS GSQMEILANQ
LTGLMDMKIF YKGKNTGSNA CVPRPCSLLC LPKANNSRSC RCPEDVSSSV
LPSGDLMCDC PQGYQLKNNT CVKQENTCLR NQYRCSNGNC INSIWWCDFD
NDCGDMSDER NCPTTICDLD TQFRCQESGT CIPLSYKCDL EDDCGDNSDE
SHCEMHQCRS DEYNCSSGMC IRSSWVCDGD NDCRDWSDEA NCTAIYHTCE
ASNFQCRNGH CIPQRWACDG DTDCQDGSDE DPVNCEKKCN GFRCPNGTCI
PSSKHCDGLR DCSDGSDEQH CEPLCTHFMD FVCKNRQQCL FHSMVCDGII
QCRDGSDEDA AFAGCSQDPE FHKVCDEFGF QCQNGVCISL IWKCDGMDDC
GDYSDEANCE NPTEAPNCSR YFQFRCENGH CIPNRWKCDR ENDCGDWSDE
KDCGDSHILP FSTPGPSTCL PNYYRCSSGT CVMDTWVCDG YRDCADGSDE
EACPLLANVT AASTPTQLGR CDRFEFECHQ PKTCIPNWKR CDGHQDCQDG
RDEANCPTHS TLTCMSREFQ CEDGEACIVL SERCDGFLDC SDESDEKACS
DELTVYKVQN LQWTADFSGD VTLTWMRPKK MPSASCVYNV YYRVVGESIW
KTLETHSNKT NTVLKVLKPD TTYQVKVQVQ CLSKAHNTND FVTLRTPEGL
PDAPRNLQLS LPREAEGVIV GHWAPPIHTH GLIREYIVEY SRSGSKMWAS
QRAASNFTEI KNLLVNTLYT VRVAAVTSRG IGNWSDSKSI TTIKGKVIPP
PDIHIDSYGE NYLSFTLTME SDIKVNGYVV NLFWAFDTHK QERRTLNFRG
SILSHKVGNL TAHTSYEISA WAKTDLGDSP LAFEHVMTRG VRPPAPSLKA
KAINQTAVEC TWTGPRNVVY GIFYATSFLD LYRNPKSLTT SLHNKTVIVS
KDEQYLFLVR VVVPYQGPSS DYVVVKMIPD SRLPPRHLHV VHTGKTSVVI
KWESPYDSPD QDLLYAVAVK DLIRKTDRSY KVKSRNSTVE YTLNKLEPGG
KYHIIVQLGN MSKDSSIKIT TVSLSAPDAL KIITENDHVL LFWKSLALKE
KHFNESRGYE IHMFDSAMNI TAYLGNTTDN FFKISNLKMG HNYTFTVQAR
CLFGNQICGE PAILLYDELG SGADASATQA ARSTDVAAVV VPILFLILLS
LGVGFAILYT KHRRLQSSFT AFANSHYSSR LGSAIFSSGD DLGEDDEDAP
MITGFSDDVP MVIA

### Seq Id No: 10

### Human Sortilin-Related Receptor 1 (sorLA) Protein

SAALQPEPI KVYGQVSLND
SHNQMVVHWA GEKSNVIVAL ARDSLALARP KSSDVYVSYD YGKSFKKISD
KLNFGLGNRS EAVIAQFYHS PADNKRYIFA DAYAQYLWIT FDFCNTLQGF
SIPFRAADLL LHSKASNLLL GFDRSHPNKQ LWKSDDFGQT WIMIQEHVKS
FSWGIDPYDK PNTIYIERHE PSGYSTVFRS TDFFQSRENQ EVILEEVRDF
QLRDKYMFAT KVVHLLGSEQ QSSVQLWVSF GRKPMRAAQF VTRHPINEYY
IADASEDQVF VCVSHSNNRT NLYISEAEGL KFSLSLENVL YYSPGGAGSD
TLVRYFANEP FADFHRVEGL QGVYIATLIN GSMNEENMRS VITFDKGGTW
EFLQAPAFTG YGEKINCELS QGCSLHLAQR LSQLLNLQLR RMPILSKESA
PGLIIATGSV GKNLASKTNV YISSSAGARW REALPGPHYY TWGDHGGIIT
AIAQGMETNE LKYSTNEGET WKTFIFSEKP VFVYGLLTEP GEKSTVFTIF
GSNKENVHSW LILQVNATDA LGVPCTENDY KLWSPSDERG NECLLGHKTV
FKRRTPHATC FNGEDFDRPV VVSNCSCTRE DYECDFGFKM SEDLSLEVCV
PDPEFSGKSY SPPVPCPVGS TYRRTRGYRK ISGDTCSGGD VEARLEGELV
PCPLAEENEF ILYAVRKSIY RYDLASGATE QLPLTGLRAA VALDFDYEHN
CLYWSDLALD VIQRLCLNGS TGQEVIINSG LETVEALAFE PLSQLLYWVD
AGFKKIEVAN PDGDFRLTIV NSSVLDRPRA LVLVPQEGVM FWTDWGDLKP
GIYRSNMDGS AAYHLVSEDV KWPNGISVDD QWIYWTDAYL ECIERITFSG
QQRSVILDNL PHPYAIAVFK NEIYWDDWSQ LSIFRASKYS GSQMEILANQ
LTGLMDMKIF YKGKNTGSNA CVPRPCSLLC LPKANNSRSC RCPEDVSSSV
LPSGDLMCDC PQGYQLKNNT CVKQENTCLR NQYRCSNGNC INSIWWCDFD
NDCGDMSDER NCPTTICDLD TQFRCQESGT CIPLSYKCDL EDDCGDNSDE
SHCEMHQCRS DEYNCSSGMC IRSSWVCDGD NDCRDWSDEA NCTAIYHTCE
ASNFQCRNGH CIPQRWACDG DTDCQDGSDE DPVNCEKKCN GFRCPNGTCI
PSSKHCDGLR DCSDGSDEQH CEPLCTHFMD FVCKNRQQCL FHSMVCDGII
QCRDGSDEDA AFAGCSQDPE FHKVCDEFGF QCQNGVCISL IWKCDGMDDC
GDYSDEANCE NPTEAPNCSR YFQFRCENGH CIPNRWKCDR ENDCGDWSDE
KDCGDSHILP FSTPGPSTCL PNYYRCSSGT CVMDTWVCDG YRDCADGSDE
EACPLLANVT AASTPTQLGR CDRFEFECHQ PKTCIPNWKR CDGHQDCQDG
RDEANCPTHS TLTCMSREFQ CEDGEACIVL SERCDGFLDC SDESDEKACS
DELTVYKVQN LQWTADFSGD VTLTWMRPKK MPSASCVYNV YYRVVGESIW
KTLETHSNKT NTVLKVLKPD TTYQVKVQVQ CLSKAHNTND FVTLRTPEGL
PDAPRNLQLS LPREAEGVIV GHWAPPIHTH GLIREYIVEY SRSGSKMWAS
QRAASNFTEI KNLLVNTLYT VRVAAVTSRG IGNWSDSKSI TTIKGKVIPP
PDIHIDSYGE NYLSFTLTME SDIKVNGYVV NLFWAFDTHK QERRTLNFRG
SILSHKVGNL TAHTSYEISA WAKTDLGDSP LAFEHVMTRG VRPPAPSLKA
KAINQTAVEC TWTGPRNVVY GIFYATSFLD LYRNPKSLTT SLHNKTVIVS
KDEQYLFLVR VVVPYQGPSS DYVVVKMIPD SRLPPRHLHV VHTGKTSVVI
KWESPYDSPD QDLLYAVAVK DLIRKTDRSY KVKSRNSTVE YTLNKLEPGG
KYHIIVQLGN MSKDSSIKIT TVSLSAPDAL KIITENDHVL LFWKSLALKE
KHFNESRGYE IHMFDSAMNI TAYLGNTTDN FFKISNLKMG HNYTFTVQAR
CLFGNQICGE PAILLYDELG SGADASATQA ARSTDVAAVV VPILFLILLS
LGVGFAILYT KHRRLQSSFT AFANSHYSSR LGSAIFSSGD DLGEDDEDAP
MITGFSDDVP MVIA

### Seq Id No: 11

### SEAP Reporter

MLLLL LLLGLRLQLS LGIIPVEEEN PDFWNREAAE ALGAAKKLQP
AQTAAKNLII FLGDGMGVST VTAARILKGQ KKDKLGPEIP LAMDRFPYVA
LSKTYNVDKH VPDSGATATA YLCGVKGNFQ TIGLSAAARF NQCNTTRGNE
VISVMNRAKK AGKSVGVVTT TRVQHASPAG TYAHTVNRNW YSDADVPASA
RQEGCQDIAT QLISNMDIDV ILGGGRKYMF RMGTPDPEYP DDYSQGGTRL
DGKNLVQEWL AKRQGARYVW NRTELMQASL DPSVTHLMGL FEPGDMKYEI
HRDSTLDPSL MEMTEAALRL LSRNPRGFFL FVEGGRIDHG HHESRAYRAL
TETIMFDDAI ERAGQLTSEE DTLSLVTADH SHVFSFGGYP LRGSSIFGLA
PGKARDRKAY TVLLYGNGPG YVLKDGARPD VTESESGSPE YRQQSAVPLD
EETHAGEDVA VFARGPQAHL VHGVQEQTFI AHVMAFAACL EPYTACDLAP
PAGTTDAAHP G

### Seq Id No: 12

### Linker 5 Amino Acids

LSRSE

### Seq Id No: 13

### Linker 8 Amino Acids

LSRSSGGE

### Seq Id No: 14

### Linker 53 Amino Acids

corresponding to sorLA propeptide (with RRKR replaced with GSGS)

EVWTQRLHGGSAPLPQDRGFLVVQGDPRELRLWARGDARGASRADEKPLG
SGS

### Seq Id No: 15

### eGluc Reporter

KPTENNEDFNIVAVASNFATTDLDADRGKLPGKKLPLEVLKEIEANARKA
GCTRGCLICLSHIKCTPKMKKFIPGRCHTYEGDKESAQGGIGEAIVDIPE
IPGFKDLEPIEQFIAQVDLCVDCTTGCLKGLANVQCSDLLKKWLPQRCAT
FASKIQGQVDKIKGAGGD

### Seq Id No: 16

### Part of sorLA Propeptide, Cleavage Site for Furin

RRKR

### Seq Id No: 17

### Substituted Cleavage Site for Furin

GSGS

### REFERENCES

Christensen S K, Endosomal trafficking is required for glycosylation and normal maturation of the Alzheimer’s-associated protein sorLA, bioRxiv preprint doi: https://doi.org/10.1101/2020.07.12.199885; Jul. 13, 2020.

Fjorback A W, Retromer binds the FANSHY sorting motif in SorLA to regulate amyloid precursor protein sorting and processing, J Neurosci. 2012 Jan 25;32(4):1467-80.

Maguire C A, Gaussia luciferase variant for high-throughput functional screening applications, Anal Chem. 2009 Aug 15;81(16):7102-6.

Mecozzi V J, Pharmacological chaperones stabilize retromer to limit APP processing, Nat Chem Biol. 2014 Jun;10(6):443-9. doi: 10.1038/nchembio.1508. Epub 2014 Apr 20.

Small S A, Endosomal traffic jams represent a pathogenic hub and therapeutic target in Alzheimer’s disease, Trends Neurosci. 2017 October; 40(10): 592-602.

Small S A, Endosomal Recycling Reconciles the Alzheimer’s Paradox, Sci Transl Med. 2020 December 02; 12(572).

Zadoo S, A Novel Luciferase Assay For Sensitively Monitoring Myocilin Variants in Cell Culture, Invest Ophthalmol Vis Sci. 2016 Apr 1;57(4):1939-50. doi: 10.1167/iovs.15-18789.

### Items

1. An assay comprising the steps of:
a. Providing cells expressing a fusion protein comprising human sortilin-related receptor 1 (sorLA) protein and a reporter fragment,
b. Contacting some sorLA-reporter fragment expressing cells with a compound and some sorLA-reporter fragment expressing cells with a buffer,
c. Determining the activity of the reporter fragment in the cell media with and without addition of the compound as a measure of sorLA-reporter fragment amount, and
d. Classifying the compound as endosomal enhancing if an increase in reporter fragment activity is observed and classifying the compound as endosomal repressing if a decrease in reporter activity is observed.

2. The method according to item 1, wherein the method is used to identify compounds that increases the amount of sorLA-reporter fusion protein in the cell media.

3. The method according to any one of the items 1 and 2, wherein the reporter fragment is selected from secreted alkaline phosphatase (SEAP), firefly (Photinus pyralis) luciferase or Renilla reniformis luciferase.

4. The method according to any one of the items 1 or 3, wherein the reporter fragment is secreted alkaline phosphatase (SEAP).

5. A fusion protein comprising human sortilin-related receptor 1 (sorLA) protein and a reporter fragment (SEQ ID NO: 1).

6. The fusion protein according to item 5, wherein, the reporter fragment is selected from the group consisting of secreted alkaline phosphatase (SEAP), firefly (Photinus pyralis) luciferase and Renilla reniformis luciferase.

7. The fusion protein according to any one of items 5 and 6, wherein the reporter fragment is secreted alkaline phosphatase (SEAP).

8. The fusion protein according to any one of items 5 to 7, wherein the reporter fragment is fused with sorLA at the N-terminal.

9. An isolated polynucleotide, encoding the fusion protein of any one of items 5 to 8.

10. A construct or a vector comprising the isolated polynucleotide according to item 9.

11. A cell comprising the isolated polynucleotide, the construct, or the vector according to any one of items 5 to 10.

12. The cell according to item 11, wherein the cell is of human origin.

13. The cell according to any one of items 11 and 12, wherein the cell is selected from the group consisting of: HeLa, HEK and SH-SY5Y cells.

14. The cell according to item 13, wherein the cell is a SH-SY5Y cell.

1. A polynucleotide construct comprising
(a) a first polynucleotide encoding sorLA protein comprising or consisting of the amino acid sequence of SEQ ID NO: 10, or a sequence variant thereof, wherein the variant is at least 80 % identical to SEQ ID NO: 10, such as at least 85%, such as at least 90%, such as at least 95%, such as at least 99%; and
(b) a second polynucleotide encoding a reporter fragment,
wherein the first and the second polynucleotides are operably linked.

2. The polynucleotide construct according to claim 1, wherein the sorLA protein is 100% identical to the amino acid sequence of SEQ ID NO: 10.

3. The polynucleotide construct according to claim 1, wherein the variant comprises a mutation prevalent in a patient suffering from Alzheimer’s disease (AD).

4. The polynucleotide construct according to claim 3, wherein the mutation is a missense mutation.

5. The polynucleotide construct according to any one of the preceding claims, wherein the reporter fragment is selected from the group consisting of Gaussia luciferase (Gluc), enhanced Gaussia luciferase (eGluc) secreted alkaline phosphatase (SEAP), firefly (Photinus pyralis) luciferase and Renilla reniformis luciferase; or optimized variants of these.

6. The polynucleotide construct according to any one of the preceding claims, wherein the reporter fragment is Gaussia luciferase (Gluc), or an optimized variant thereof.

7. The polynucleotide construct according to any one of the preceding claims, wherein the reporter fragment is enhanced Gaussia luciferase (eGluc).

8. The polynucleotide construct according to any one of the preceding claims, wherein the reporter fragment is 100% identical to the amino acid sequence of SEQ ID NO: 15.

9. The polynucleotide construct according to any one of the preceding claims, wherein the reporter fragment is selected from the group consisting of Gluc-M431l, Gluc-M110l and GlucM43l.

10. The polynucleotide construct according to any one of the preceding claims, wherein the reporter fragment is secreted alkaline phosphatase (SEAP), or an optimized variant thereof.

11. The polynucleotide construct according any one of the preceding claims, wherein the reporter fragment is 100% identical to the amino acid sequence of SEQ ID NO: 11.

12. The polynucleotide construct according to any one of the preceding claims, wherein upon expression the reporter fragment is fused with sorLA at the N-terminal of sorLA.

13. The polynucleotide construct according to any one of the preceding claims, further comprising a third polynucleotide encoding a linker, wherein the first, the second and the polynucleotides are operably linked.

14. The polynucleotide construct according to any one of the preceding claims, wherein the linker links the sorLA protein to the reporter fragment.

15. The polynucleotide construct according to any one of the preceding claims, wherein upon expression the linker is fused with sorLA at the N-terminal of sorLA.

16. The polynucleotide construct according to any one of the preceding claims, wherein upon expression the reporter fragment is fused with the linker at the N-terminal of the linker.

17. The polynucleotide construct according to any one of the preceding claims, wherein the linker consists of at least one amino acid, for example 2 amino acids, such as 3 amino acids, for example 4 amino acids, such as 5 amino acids, for example 6 amino acids, such as 7 amino acids, for example 8 amino acids, such as 9 amino acids, for example 19 amino acids, such as 11 amino acids, for example 12 amino acids, such as 13 amino acids, for example 14 amino acids, such as 15 amino acids, for example 16 amino acids, such as 17 amino acids, for example 18 amino acids, such as 19 amino acids, for example 20 amino acids, such as 25 amino acids, for example 30 amino acids, such as 35 amino acids, for example 40 amino acids, such as 45 amino acids, for example 50 amino acids, such as 55 amino acids, for example 60 amino acids, such as 65 amino acids, for example 70 amino acids, such as 75 amino acids, for example 80 amino acids, such as 85 amino acids, for example 90 amino acids, such as 95 amino acids, for example 100 amino acids.

18. The polynucleotide construct according to any one of the preceding claims, wherein the linker consists of 2 amino acids.

19. The polynucleotide construct according to any one of the preceding claims, wherein the linker consists of 5 amino acids.

20. The polynucleotide construct according to any one of the preceding claims, wherein the linker consists of 8 amino acids.

21. The polynucleotide construct according to any one of the preceding claims, wherein the linker consists of 53 amino acids.

22. The polynucleotide construct according to any one of the preceding claims, wherein the linker consists of an amino acid sequence of LE.

23. The polynucleotide construct according to any one of the preceding claims, wherein the linker consists of an amino acid sequence of LSRSE (SEQ ID NO: 12).

24. The polynucleotide construct according to any one of the preceding claims, wherein the linker consists of an amino acid sequence of LSRSSGGE (SEQ ID NO: 13).

25. The polynucleotide construct according to any one of the preceding claims, wherein the linker consists of an amino acid sequence of EVWTQRLHGGSAPLPQDRGFLVVQGDPRELRLWARGDARGASRADEKPLGSG S (SEQ ID NO: 14).

26. The polynucleotide construct according to any one of the preceding claims, encoding a fusion protein comprising or consisting of an amino acid sequence of SEQ ID NO: 6.

27. The polynucleotide construct according to any one of the preceding claims, encoding a fusion protein comprising or consisting of an amino acid sequence of SEQ ID NO: 7.

28. The polynucleotide construct according to any one of the preceding claims, encoding a fusion protein comprising or consisting of an amino acid sequence of SEQ ID NO: 8.

29. The polynucleotide construct according to any one of the preceding claims, encoding a fusion protein comprising or consisting of an amino acid sequence of SEQ ID NO: 9.

30. The polynucleotide construct according to any one of the preceding claims, encoding a fusion protein comprising or consisting of an amino acid sequence of SEQ ID NO: 1.

31. The polynucleotide construct according to any one of the preceding claims, encoding a fusion protein comprising or consisting of an amino acid sequence of SEQ ID NO: 2.

32. The polynucleotide construct according to any one of the preceding claims, encoding a fusion protein comprising or consisting of an amino acid sequence of SEQ ID NO: 3.

33. The polynucleotide construct according to any one of the preceding claims, encoding a fusion protein comprising or consisting of an amino acid sequence of SEQ ID NO: 4.

34. The polynucleotide construct according to any one of the preceding claims, encoding a fusion protein comprising or consisting of an amino acid sequence of SEQ ID NO: 5.

35. A vector comprising the polynucleotide construct according to any one of the preceding claims.

36. The vector according to claim 35, wherein the vector is selected from the group consisting of plasmids, viral vectors and cosmids.

37. The vector according any one of claims 35 to 36, wherein the vector is a plasmid vector.

38. The vector according any one of claims 35 to 37, wherein the vector is a viral vector.

39. The vector according any one of claims 35 to 38, wherein the vector is functional in mammalian cells.

40. The vector according any one of claims 35 to 39, wherein the vector further comprises 5′ and 3′ terminal repeats.

41. The vector according to any one of claims 35 to 40, wherein the vector comprises a promoter.

42. The vector according to any one of claims 35 to 41, wherein said promoter is specific for mammalian cells.

43. The vector according to any one of claims 35 to 42, wherein the mammalian cell is a neural cell.

44. The vector according to any one of claims 35 to 43, wherein the vector further comprises a polyadenylation sequence.

45. The vector according to any one of claims 35 to 44, wherein said nucleotide sequence is operably linked to a post-transcriptional regulatory element.

46. A fusion protein encoded by the polynucleotide construct according to any one of claims 1 to 34 or by the vector according to any one of claims 35 to 45.

47. The fusion protein according to claim 46, wherein the fusion protein comprises or consists of an amino acid sequence of SEQ ID NO: 6.

48. The fusion protein according to claim 46, wherein the fusion protein comprises or consists of an amino acid sequence of SEQ ID NO: 7.

49. The fusion protein according to claim 46, wherein the fusion protein comprises or consists of an amino acid sequence of SEQ ID NO: 8.

50. The fusion protein according to claim 46, wherein the fusion protein comprises or consists of an amino acid sequence of SEQ ID NO: 9.

51. The fusion protein according to claim 46, wherein the fusion protein comprises or consists of an amino acid sequence of SEQ ID NO: 1.

52. The fusion protein according to claim 46, wherein the fusion protein comprises or consists of an amino acid sequence of SEQ ID NO: 2.

53. The fusion protein according to claim 46, wherein the fusion protein comprises or consists of an amino acid sequence of SEQ ID NO: 3.

54. The fusion protein according to claim 46, wherein the fusion protein comprises or consists of an amino acid sequence of SEQ ID NO: 4.

55. The fusion protein according to claim 46, wherein the fusion protein comprises or consists of an amino acid sequence of SEQ ID NO: 5.

56. A cell comprising the polynucleotide construct according to any one of claims 1 to 34 and/or the vector according to any one of claims 35 to 45 and/or the fusion protein according to any one of claims 46 to 55.

57. The cell according to claim 56, wherein the cell is of mammalian origin.

58. The cell according any one of claims 56 to 57, wherein the cell is of human origin.

59. The cell according any one of claims 56 to 58, wherein the cell is selected from the group consisting of HeLa, HEK and SH-SY5Y cells.

60. The cell according any one of claims 56 to 59, wherein the cell is a SH-SY5Y cell.

61. A method for classifying a compound as endosomal enhancing or endosomal repressing, the method comprising the steps of:
(a) Providing cells according to any one of claims 56 to 60,
wherein the cells express a fusion protein according to any one of claims 46 to 55 consisting or comprising of a sorLA-reporter fragment;
(b) Contacting a first aliquot of cells of (a) with medium comprising a compound, and contacting a second aliquot of cells of (a) with medium not comprising a compound,
(c) Determining and comparing the activity of the sorLA-reporter fragment in the first and second aliquot, and
(d) Classifying the compound as
i. endosomal enhancing if an increase in reporter fragment activity is observed in the first aliquot of cells compared to the second aliquot of cells, or
ii. endosomal repressing if a decrease in reporter fragment activity is observed in the first aliquot of cells compared to the second aliquot of cells.

62. The method according to claim 61, wherein the method is used to identify compounds that increase the amount of sorLA-reporter fusion protein in the cell media.

63. The method according to claim 61, wherein the method is used to identify compounds that decrease the amount of sorLA-reporter fusion protein in the cell media.

64. The method according any one of claims 61 to 63, wherein the classified compound is
- suitable for use in the treatment of Alzheimer’s disease (AD) if the compound is classified as endosomal enhancing, or
- unsuitable for use in the treatment of Alzheimer’s disease (AD) if the compound is classified as endosomal repressing.

65. A method for classifying a sorLA mutation as endosomal enhancing or endosomal repressing, the method comprising the steps of:
(a) Providing cells according to any one of claims 56 to 60, wherein the cells express a fusion protein according to any one of claims 46 to 55 consisting or comprising of a sorLA-reporter fragment, and wherein the sorLA protein is 100% identical to the amino acid sequence of SEQ ID NO: 10, and
(b) Providing cells according to any one of claims 56 to 60, wherein the cells express a fusion protein according to any one of claims 46 to 55 consisting or comprising of a sorLA-reporter fragment, and wherein the sorLA protein is a variant comprising a mutation prevalent in a patient suffering from Alzheimer’s disease (AD),
(c) Culturing the cells of (a) and (b),
(d) Determining and comparing the activity of the sorLA-reporter fragment in medium from cells of (a) and (b), and
(e) Classifying the sorLA mutation as
i. endosomal enhancing if an increase in reporter fragment activity is observed in the medium of the cells of (b) compared to the medium of the cells of (a), or
ii. endosomal repressing if a decrease in reporter fragment activity is observed in the medium of the cells of (b) compared to the medium of the cells of (a).

66. A method for testing if a patient identified with a SORL1 mutation will benefit from treatment with a compound, the method comprising the steps of:
(a) Providing cells according to any one of claims 56 to 60, wherein the cells express a fusion protein according to any one of claims 46 to 55 consisting or comprising of a sorLA-reporter fragment, and wherein the sorLA protein is a variant comprising said SORL1 mutation,
(b) Contacting a first aliquot of cells of (a) with medium comprising a compound, and contacting a second aliquot of cells of (a) with medium not comprising a compound,
(c) Determining and comparing the activity of the sorLA-reporter fragment in the first and second aliquot, and
(d) Concluding that the patient will
i. benefit from treatment with the compound if an increase in reporter fragment activity is observed in the first aliquot of cells compared to the second aliquot of cells, or
ii. not benefit from treatment with the compound if a decrease in reporter fragment activity is observed in the first aliquot of cells compared to the second aliquot of cells.

67. The methods according to any one of claims 61 to 66, wherein determining the activity of the substrate comprises the addition of a substrate for the reporter fragments.

68. A kit for conducting a screening assay for classifying a compound as endosomal enhancing or endosomal repressing, the kit comprising
-the polynucleotide construct according to any one of claims 1 to 34, and/or
-the vector according to any one of claims 35 to 45, and/or
-the cell according to any one of claims 46 to 55; and instructions for use.

69. The methods or the kit according to any one of claims 61 to 68, wherein the compound is selected from the group consisting of small molecules, a peptides, polypeptides, macromolecules, a lipids, siRNAs, microRNAs and cDNAs.

70. The methods or the kit according to any one of claims 61 to 69, wherein the compound is derived from CRISPR/Cas9 arrayed libraries.
